SEQUENCE LISTING (14 sequences)

SEQ ID NO: 1 - protein, 37 aa, Artificial Sequence
  synthetic
KWKLFKKIEKVGQNIRDGIIKAGPAVAVVGQATQIAK

SEQ ID NO: 2 - protein, 8 aa, Artificial Sequence
  synthetic
KWKLFKKI

SEQ ID NO: 3 - protein, 5 aa, Artificial Sequence
  synthetic
  DISULFID (1)..(5): cyclic peptide
CNGRC

SEQ ID NO: 4 - protein, 8 aa, Artificial Sequence
  synthetic
VPMSMRGG

SEQ ID NO: 5 - protein, 8 aa, Artificial Sequence
  synthetic
IPVSLRSG

SEQ ID NO: 6 - protein, 14 aa, Artificial Sequence
  synthetic
GGGGPLGVRGGGGK

SEQ ID NO: 7 - protein, 8 aa, Artificial Sequence
  synthetic
RPFSMIMG

SEQ ID NO: 8 - protein, 8 aa, Artificial Sequence
  synthetic
VPLSLTMG

SEQ ID NO: 9 - protein, 8 aa, Artificial Sequence
  synthetic
VPLSLYSG

SEQ ID NO: 10 - protein, 8 aa, Artificial Sequence
  synthetic
IPESLRAG

SEQ ID NO: 11 - protein, 20 aa, Artificial Sequence
  synthetic
AEGEFCSWAPPKASCGDPAK

SEQ ID NO: 12 - protein, 11 aa, Artificial Sequence
  synthetic
CSWRPPFRAVC

SEQ ID NO: 13 - protein, 11 aa, Artificial Sequence
  synthetic
CSWAPPFKASC

SEQ ID NO: 14 - protein, 11 aa, Artificial Sequence
  synthetic
CNWTPPFKTRC

(19) United States
(12) Patent Application Publication
(10) Pub. No.: US 2020/0164086 A1
(43) Pub. Date: May 28, 2020

(54) FUSOGENIC LIPOSOMES, COMPOSITIONS, KITS AND USE THEREOF FOR TREATING CANCER

(71) Applicant: APA- ADVANCED TECHNOLOGIES LTD., TEL-AVIV (IL)

(72) Inventors: Igor NUDELMAN, Haifa (IL); Yael LUPU-HABER, Haifa (IL); Galoz KANETI, Haifa (IL); David GERSHON, Kiryat Tiv'on (IL); Haim ALCALAY, Tel-Aviv (IL); Avi SCHROEDER, Binyamina (IL)

(21) Appl. No.: 16/657,347

(22) Filed: Oct. 18, 2019

Related U.S. Application Data
(63) Continuation-in-part of application No. PCT/IL2018/050434, filed on Apr. 17, 2018.
(60) Provisional application No. 62/638,408, filed on Mar. 5, 2018.
(60) Provisional application No. 62/487,105, filed on Apr. 19, 2017.

(51) Int. Cl. A61K 47/69; A61P 35/00; A61K 47/68

(52) U.S. Cl. CPC A61K 47/6911; A61K 47/6913; A61K 47/6849; A61P 35/00

(57) ABSTRACT

A fusogenic liposome comprising a lipid bilayer comprising a plurality of lipid molecules having 14 to 24 carbon atoms, wherein at least one of said lipid molecules is functionalised with a first functional group of a specific binding pair capable of binding to a complementary second functional group of said binding pair; and optionally further comprising an immune system activating agent functionalised with a complementary second functional group of said binding pair bound to said first functional group is provided. Methods of treatment of cancer using the fusogenic liposome are also provided.

### CROSS-REFERENCE TO RELATED APPLICATIONS

The present application is a Continuation in Part of PCT/IL2018/050434, filed Apr. 17, 2018, the entire contents of which being hereby incorporated herein by reference. The present application also claims benefit to U.S. application 62/638,408 and U.S. application 62/487,105, the entire contents of each of which being hereby incorporated herein by reference.

### SEQUENCE LISTING

The Sequence Listing in ASCII text file format of 3,511 bytes in size, created on Dec. 24, 2019, with the file name “2020-02-11SequenceListing-NUDELMAN5A.txt,” filed in the U.S. Patent and Trademark Office on Feb. 11, 2020, is hereby incorporated herein by reference.

### FIELD OF THE INVENTION

The present invention relates in general to supramolecular assemblies including liposome constructs for use in cancer therapy.

### BACKGROUND OF THE INVENTION

Immunotherapy is considered one of the most promising areas in cancer therapy, since it harnesses the body's own immune system to fight cancer^{1,2}. It was suggested³ that the tumor formation starts with heterogenic tumor cells (containing sufficient driver and passenger mutations)⁴ which are attacked by immune cells⁵, resulting in a less heterogenic tumor niche⁶ which then evades effective identification by immune cells and recruits anti-inflammatory cells such as regulatory T cells and tumor infiltrating macrophages (TAMS)⁷. Different cancer types employ mechanisms that allow it to evade immune detection and consequently to escape killing by immune cells. The alternatives, which are usually employed as first line of treatment, chemotherapy, usually include off target side effects that gravely impact the patients' quality of life from damage to mucosa, skin, bone marrow and other tissues as well. Predominant anti-cancer immune therapies include chimeric antigen receptor-T (CAR-T) cells⁸, tumor infiltrating leukocytes (TIL) used against primary and metastatic cancers⁹, and immune checkpoint blockage using inhibition blocking antibodies¹⁰. These approaches depend on cancer cell identification by the immune cells to allow cancer killing⁷. For instance, the CAR-T cell approach requires an existing marker on the cancer cell, (e.g. Yescarta™ and CD19 positive cancer cells), TIL approach requires a high abundance of tumor associated mutations and the immune checkpoint blockade requires high cancer expression levels of inhibitory molecules (e.g. PD1L/PD2L levels above 50%)^{3,11} These approaches hold several severe side effects that are long-lived. The CAR-T cells requires isolation and transfection of the T cells to express an engineered T cell receptor with a cancer binding protein (single chain FV, SCfv), grow and expand them under ex-vivo conditions. Furthermore, CAR-T lack an “off-switch” to allow patients suffering from severe side effects to improve overall wellbeing. The TIL approach requires tumor biopsy to allow isolation of T cells, their activation, expansion and re-insertion into the patients¹. Both CAR-T cell and TIL approaches promote anti-cancer immune activity. Immune checkpoint inhibitors, allow immune cells to attack an immune evasive cancer, expressing elevated levels of PD1L for example. By employing anti-PD1 therapy, PD1 mediated inhibitory signal is hindered in a systemic manner and was shown to include auto-immune side effects. All of the abovementioned approaches require killer T cell identification of cancer cells as a pre-requisite for efficacy (i.e. cancer cells must present peptides that killer T cell recognizes and therefore is activated and kills cancer cell).

There remains therefore an urgent need for techniques that circumvent the limited natural selection of tumor-specific antigens while utilizing the immune systems' natural ability to attack and kill tumor cells.

### SUMMARY OF INVENTION

The present application describes embodiments of an immune labelling platform that allows killing of cancer cells by specifically activating the immune system. The concept includes modifying cell membranes to label specific cells with immune activating agents by the use of lipids capable of integrating into or reacting with a target cell, wherein the lipids form assemblies such as liposomes, micelles, and cubosomes, which can fuse with membranes; and a supramolecular assembly designed for releasing lipids within or in the vicinity of a tumor, such as a lipid gel, a lipid sponge, a bilayer or monolayer lipid sheet, a filamentous lipid structure and a lipid cochleate. Lipid particles reacting with a target cell refers to a reaction of reactive groups found on the supramolecular assembly or on the immune-system activating agent with reactive groups on the surface of the cell (like amines of proteins on the surface or others reactive groups). For example, Tosyl-PEG4-Azide could react upon release from liposomes with proteins on the surface of cancer/target cells.

In a non-limiting example, the liposomes of the present invention fuse with cancer cells and result in the exhibition of the antibodies on the cancer cells' membranes. We add a new target on the cancer cells that allows the killer T cells to recognize the cancer cells. The immune-labelled cancer cell binds e.g. an effector/memory killer T cell specific for a specific viral/non-self-peptide. The antibody-set activates the T cell, which kills the cancer cell, secretes pro-inflammatory cytokines (IL2, IFNγ etc.) and starts clonal expansion. The resulting clones are effector killer T cells that are specific only for killing of the same specific viral/non-self-peptide presenting cells. The expanded T cells will look for such cells and will only kill those or liposomal labelled cells.

Alternatively, when the liposome treated cancer cell meets a naïve killer T cell specific for a “self” peptide, the antibody set binds the naïve killer T cell via T cell receptor. Since the naïve T cell was not legitimately activated (by an antigen presenting cell with co-stimulatory molecules), it will undergo anergy, which means that it will not be activated and therefore unable to kill, secrete pro-inflammatory cytokines or become activated.

In addition to the retention enhanced permeability (RES) effect, which improves arrival of liposomes to tumors¹², we use the intrinsic general negative charge on the tumor cells as opposed to the neutral nature of the healthy cells¹³⁻¹⁵ as an selectivity enhancing element in our liposomal platform.

In one aspect, the present invention provides a supramolecular assembly comprising a plurality of lipids, wherein the hydrophilic head of at least one lipid of the supramolecular assembly is functionalised with a functional group or with one or more immune-system activating agents. This functional group is a member of a binding pair, such as thiol-maleimide, azide-alkyne, aldehyde-hydroxylamine etc.

In another aspect, the present invention provides a fusogenic liposome comprising a lipid bilayer comprising a plurality of lipid molecules having 14 to 24 carbon atoms, wherein at least one of said lipid molecules is functionalised with a first functional group of a specific binding pair capable of binding to a complementary second functional group of said binding pair.

In an additional aspect, the present invention provides method for preparation of a fusogenic liposome with an immune system activating agent bound at the outer leaflet, said method comprising the reaction of a functionalised fusogenic liposome comprising a lipid bilayer comprising a plurality of lipid molecules having (a) 14 to 24 carbon atoms and (b) a first functional group of a specific binding pair capable of binding to a complementary second functional group of said binding pair with an immune system activating agent functionalised with a complementary second functional group of the binding pair, wherein said second functional group binds to said first functional group, thereby yielding said fusogenic liposome with said immune system activating agent bound at the outer leaflet.

In yet an additional aspect, the present invention provides a method for preparation of a fusogenic liposome with an immune system activating agent bound at both the inner and outer leaflet, said method comprising the steps of (i) reacting a plurality of lipid molecules having 14 to 24 carbon atoms, wherein at least one of said lipid molecules is functionalised with a first functional group of a specific binding pair, with an immune system activating agent functionalised with a second functional group of the binding pair, wherein said second functional group binds to said first functional group of said lipid molecules, thereby yielding the lipid molecules linked to the immune system activating agent; and (ii) preparing said fusogenic liposome from said lipid molecules obtained in step (i), thereby yielding the fusogenic liposome functionalised with said immune system activating agent bound at both the inner and outer leaflet.

In still an additional aspect, the present invention provides a method for preparation of a fusogenic liposome with an immune system activating agent bound at the inner leaflet with a practically minimum of immune system agent bound at the outer leaflet.

In another aspect, the present invention provides a method for treating cancer by labelling cancer cells with an immune-system activating agent, said method comprising administering to a cancer patient a fusogenic liposome, wherein the method comprises the steps of (i) administering to said cancer patient an immune-system activating fusogenic liposome comprising: (a) a lipid bilayer comprising a plurality of lipid molecules having 14 to 24 carbon atoms, and a first functional group of a specific binding pair capable of binding to a complementary second functional group of said binding pair; and (b) an immune-system activating agent comprising said complementary second functional group of said binding pair bound to said first functional group; or (ii) administering to said cancer patient a functionalised fusogenic liposome comprising a lipid bilayer comprising a plurality of lipid molecules having 14 to 24 carbon atoms, wherein at least one of said lipid molecules is functionalised with a first functional group of a specific binding pair capable of binding to a complementary second functional group of said binding pair; and subsequently to step (ii) administering an immune-system activating agent functionalised with a complementary second functional group of the binding pair capable of binding to said first functional group of said lipid molecules.

In a further aspect, the present invention provides a kit comprising (a) a first container comprising a fusogenic liposome comprising (a) a lipid bilayer comprising a plurality of lipid molecules having 14 to 24 carbon atoms, wherein at least one of said lipid molecules is functionalised with a first functional group of a specific binding pair capable of binding to a complementary second functional group of said binding pair; (b) a second container comprising a T-cell activating agent functionalised with a second functional group of the binding pair capable of binding to said first functional group of said lipid molecules; and (c) a pamphlet with instructions for a method for treating cancer comprising administering to a cancer patient the fusogenic liposome of (a) and subsequently the T-cell activating agent of (b).

In yet an additional aspect, the present invention provides a pharmaceutical composition comprising the fusogenic liposome as defined in any one of the above embodiments and a pharmaceutically acceptable carrier.

Various embodiments may allow various benefits, and may be used in conjunction with various applications. The details of one or more embodiments are set forth in the accompanying figures and the description below. Other features, objects and advantages of the described techniques will be apparent from the description and drawings and from the claims.

### BRIEF DESCRIPTION OF DRAWINGS

The patent or application file contains at least one drawing executed in color. Copies of this patent or patent application publication with color drawings will be provided by the Office upon request and payment of the necessary fee.

Disclosed embodiments will be understood and appreciated more fully from the following detailed description taken in conjunction with the appended figures. The drawings included and described herein are schematic and are not limiting the scope of the disclosure. It is also noted that in the drawings, the size of some elements may be exaggerated and, therefore, not drawn to scale for illustrative purposes. The dimensions and the relative dimensions do not necessarily correspond to actual reductions to practice of the disclosure.

FIG. 1A schematically shows: on the left, a lipid molecule forming a lipid bilayer and modified with a first functional group F₁; and on the right, an immune-system activating agent modified with a second functional group F₂.

FIG. 1B schematically shows: on the left, a lipid molecule forming a lipid bilayer and modified with a first crosslinker comprising a first functional group F₁ and a first spacer; and on the right, an immune-system activating agent modified with a second crosslinker comprising a second functional group F₂ and a second spacer.

FIGS. 2A-F shows mode of action and liposomal immuno-labelling of cancer cells. A. Liposomal platform layout: binding pair (I) is covalently linked to linker molecule (II) and connected to lipid head group. Modified lipid is used alongside other fusion enhancing lipids to comprise liposome. B. Monoclonal antibody (mAb) with linker containing one functional group of binding pair (I) attached to linker with other binding pair (II), covalently connected to lipid head (III). C. Linker attachment example to antibody and to lipid head group: NHS is added to one end of the linker and azide or BCN (bicyclo[6.1.0]non-4-yne) is added to the other end of the linker resulting in two “clickable” crosslinkers. The primary amine (from diacyl-phosphatidyl-ethanolamine or lysine side group from antibody) attacks the NHS (leaving group, LG), and labels the lipid head group and antibody with clickable linkers. The azide and BCN reacts resulting in covalent bond formation between the lipid head and antibody via the linker formed by the two clickable crosslinkers. D. Fusion versus uptake assay: a FITC (green fluorescence) labeled liposome (formulation N8: DOTAP:DOPC:DOPE:DOPE-FITC:DSPE-PEG2K 35:52.5:10:0.2:X where X is 5, 2.5, 1.25, 0.625, molar ratio) was used with azide bound PEG linker (194 Da). Liposomes were incubated with cancer cells at 0.5 mM lipids, washed and stained using a red fluorescent probe labelled with DBCO (dibenzocyclooctyne)-Cy5 that is reactive with azide. E. Illustration of different approaches used to deliver T cell activating antibodies to tumors: IN (I) approach is achieved by binding the mAb to the inner leaflet of the liposome and achieved using a copper-dependent click reaction to allow removal of reagents, catalysts and unbound mAbs during production process. OUT (II) approach is achieved by binding the mAb to the outer leaflet of the liposomes after liposome production. IN/OUT approach is achieve by binding the mAb to both the inner and outer leaflets of the liposome. F. Immune labelling of cancer cell—mode of action: N8 liposomes (DOTAP:DOPC: DOPE:DOPE-FITC:DSPE-PEG2K 35:52.5:10:0.2:2.5, molar ratio) were used with azide bound PEG₄ linker (194 Da). Liposomes were incubated with cancer cells at 5 mM lipids for 1 hr, washed and labeled using anti-CD3-PEG₄-BCN and anti-CD8-PEG₄-BCN for 1 hr and washed. Immune labeled cancer cells activate killer T cells and are killed (illustrated by degranulation red arrow and red dots).

FIGS. 3A-C show calibration of preparation of Calcein conjugated liposomes. A. Thin layer chromatography of synthesis of 6-Heptynoic-PE for liposome conjugation via ‘click’ chemistry. B. The effect of the length of the phospholipid hydrophobic tail on liposome cell uptake (37° C.) and liposome cell fusion (4° C.). C. The effect of the cholesterol concentration in liposome formulation on liposome cell uptake (37° C.) and liposome cell fusion (4° C.).

FIG. 4 shows liposomal composition activity study: fusion with cancer cells. DSPE\DOPE-PEG4-N3 modified liposomes or control liposomes (unmodified DSPE) were incubated with 4T1mCherry cells for 1 hr 37° C. at 0.5 mM lipids, washed and stained using DBCO-Cy5 following analyses using flow cytometry (orange and blue respectively). Presented are the averaged percent of Cy5-labeled cells for each liposomal composition. Error bars represent standard deviation.

FIGS. 5A-B percent fluorescence-positive cells and mean fluorescent intensity of a cancer cell panel treated with N8 formulation using different DSPE-PEG2000 ratios compared with DOXIL and unlabeled (no FITC and no azide) liposomes. A. Liposome uptake and fusion mediated labeling were determined for different cancer cell lines and are presented as percent of gated cells positive for fluorescent signal. B. The mean fluorescent index/intensity (MFI) of liposome treated cells is presented. The increase in fluorescence is proportional to the number of fluorescent groups in or on the cancer cells. Average of fluorescently labeled cells out of total gated cells (10,000 per tube, in triplicates, in two independent repeats) is presented in bar charts. Error bars represent standard deviation.

FIG. 6 shows Z stack of 4T1mCherry cells treated with FITC labeled liposomes. 4T1mCherry cells were incubated with (DOTAP:DOPC:DOPE:DOPE-FITC: DSPE-PEG2K 35:52.5:10:0.2:2.5) at 5 mM lipids for 1 hr at 37° C. Nuclei were stained using Hoechst. Cells were washed and imaged using confocal laser scanning microscope (LSM 710). Scale bar represents 20 μm.

FIGS. 7A-C show liposomes target cytoplasmic membranes of cancer cells. A. A549 human lung cancer cells, were stained using PKH26 dye prior to experiment and were incubated with liposomes (DOTAP:DOPC:DOPE:DOPE-FITC:DSPE-PEG2K 35:52.5:10:0.2:2.5) at 5 mM lipids for 1 hr. Nuclei were stained using Hoechst (blue). Cells were washed and imaged using confocal laser scanning microscope (LSM 710). B. Louis Lung carcinoma (murine) were treated identically to cells in B. C. B16 murine melanoma cells were treated identically to cells in A. scale bar 20 μm.

FIG. 8 shows confocal time lapse of immune labeling liposomes treated 4T1mCherry cells, co-incubated with killer T cells.4T1mCherry cells (red, larger adherent cells—seen as light gray in gray scale since each channel is separated into a different column) treated with 5 mM lipids for 1 hr and supplemented with anti-CD3 and anti-CD8, modified using PEG4-BCN (2STEP approach), were co-incubated with CFSE (green) labeled primary killer T cells (smaller, non-adherent cells). Co-culture was maintained at 5% CO₂, at 37° C. Presented are confocal images taken every 50 minutes. Scale bar represents 20 μm.

FIG. 9 shows confocal time lapse of untreated 4T1mCherry cells, co-incubated with killer T cells. 4T1mCherry cells (red, larger adherent cells—seen as light gray in gray scale since each channel is separated into a different column) were co-incubated with CFSE labeled primary killer T cells. Co-culture was maintained at 5% CO₂, at 37° C. Presented are confocal images taken every 50 minutes. Scale bar represents 20 μm.

FIG. 10 shows image analysis of red pixel percent in confocal time lapse images. Presented are the percent of red pixels in images taken from time laps at different time points (0, 300, 600 and 900 minutes) shown in FIGS. 8 and 9. Percent of red pixels (cancer cell signal) are presented for 2STEP (black circles) or for untreated control (gray circles). Image quantification was performed using FIJI image analysis software under identical parameters.

FIGS. 11A-C shows systemic efficacy and biodistribution of immune labeling liposomes in triple negative breast cancer mouse model. Two approaches were compared in tumor bearing mice; one-step and two-step approaches. Liposomal formulations were injected I.V. on day 3 and day 10 (red arrows). 2STEP-labeling liposomes comprising DOPE-PEG₄-BCN were injected and 3 hrs post injection “clickable” mAbs (mAbs labeled with PEG-azide) were injected I.V.; 1STEP: IN-mAbs linked to inner leaflet; or IN+OUT-mAbs linked to both inner and outer leaflets; 2STEP control-contains the same lipid formulation as 2STEP but with unlabeled mAbs. A. Tumor size averages and error bars (standard error) are presented. B. Individual spider plots (each graph presents one group, each series presents tumor size data from one mouse) are presented. C. Biodistribution of N8 liposomes (DOTAP:DOPC:DOPE:DSPE-PEG2000 35:52.5:10:2.5 where DOPE was modified using PEG-azide (N8) or with anti-CD3 and anti-CD8 (N8+OUT)) with or without anti-CD3 and anti-CD8 mAbs bound to outer leaflet (OUT) compared with DOXIL formulation at 24 hrs post injection.

FIGS. 12A-C show immuno-histochemical and histological analyses of tumor, kidney and livers isolated from animals at 72 hrs from immune labeling liposomes. A. Tumors, kidneys and livers were isolated, neutral base formalin fixed, embedded in paraffin, and stained using hematoxylin and eosin (H&E) or with anti-CD3. H&E stains are generally used to detect changes in tissue morphology which indicates tissue damage. Anti-CD3 staining was used to detect T cells in the selected tissues (dark brown, some highlighted with green arrow heads). Inset on the left of each micrograph represent the slide overview. B. Tumors, kidneys, livers, lungs and spleens were isolated from 4T1 tumor bearing mice and were digested into single cells. Cells were incubated with 0.5 mM lipids of N8 formulation (2STEP or OUT) for 1 hr and were washed and stained using DBCO-Cy5. Primary cells from 2 mice at triplicates were analyzed using flow cytometry for Cy5 fluorescence. C. Brown pixels (T cell signal) were quantified using FIJI and are presented as percent of tested region of interest (ROI). Tumor, liver and kidney images were divided into at least 10 ROIs (approximately 100 μm², excluding tumor necrotic core) and analyzed for CD3 staining. Error bars represent standard deviation.

### DETAILED DESCRIPTION OF THE INVENTION

In the following description, various aspects of the present application will be described. For purposes of explanation, specific configurations and details are set forth in order to provide a thorough understanding of the present application. However, it will also be apparent to one skilled in the art that the present application may be practiced without the specific details presented herein. Furthermore, well-known features may be omitted or simplified in order not to obscure the present application.

The term “comprising”, used in the claims, is “open ended” and means the elements recited, or their equivalent in structure or function, plus any other element or elements which are not recited. It should not be interpreted as being restricted to the means listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a device comprising x and z” should not be limited to devices consisting only of components x and z. Also, the scope of the expression “a method comprising the steps x and z” should not be limited to methods consisting only of these steps.

Unless specifically stated, as used herein, the term “about” is understood as within a range of normal tolerance in the art, for example within two standard deviations of the mean. In one embodiment, the term “about” means within 10% of the reported numerical value of the number with which it is being used, preferably within 5% of the reported numerical value. For example, the term “about” can be immediately understood as within 10%, 9%, 8%, 7%, 6%, 5%, 4%, 3%, 2%, 1%, 0.5%, 0.1%, 0.05%, or 0.01% of the stated value. In other embodiments, the term “about” can mean a higher tolerance of variation depending on for instance the experimental technique used. Said variations of a specified value are understood by the skilled person and are within the context of the present invention. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges, for example from 1-3, from 2-4, and from 3-5, as well as 1, 2, 3, 4, 5, or 6, individually. This same principle applies to ranges reciting only one numerical value as a minimum or a maximum. Unless otherwise clear from context, all numerical values provided herein are modified by the term “about”. Other similar terms, such as “substantially”, “generally”, “up to” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value.

As used herein, the term “and/or” includes any and all combinations of one or more of the associated listed items. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The supramolecular assembly of the invention, its preparation and use thereof, are described with respect to the following aspects, sentences, and embodiments. Unless otherwise stated, all aspects, sentences and embodiments are to be read as being able to be combined with any other aspects, sentences and embodiments, unless such combination would not make technical sense or is explicitly stated otherwise. For clarity, the specific embodiments of the supramolecular assembly are defined in the context of the specific configuration of fusogenic liposomes, but they are applicable also to the other configurations of the supramolecular assembles.

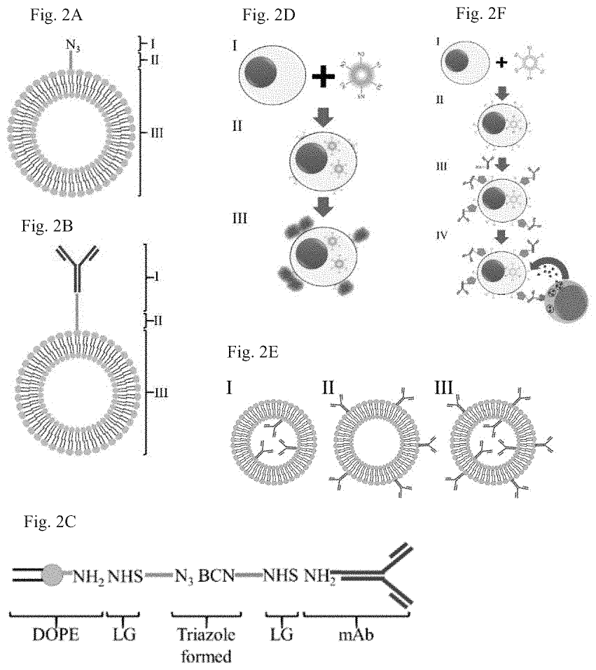

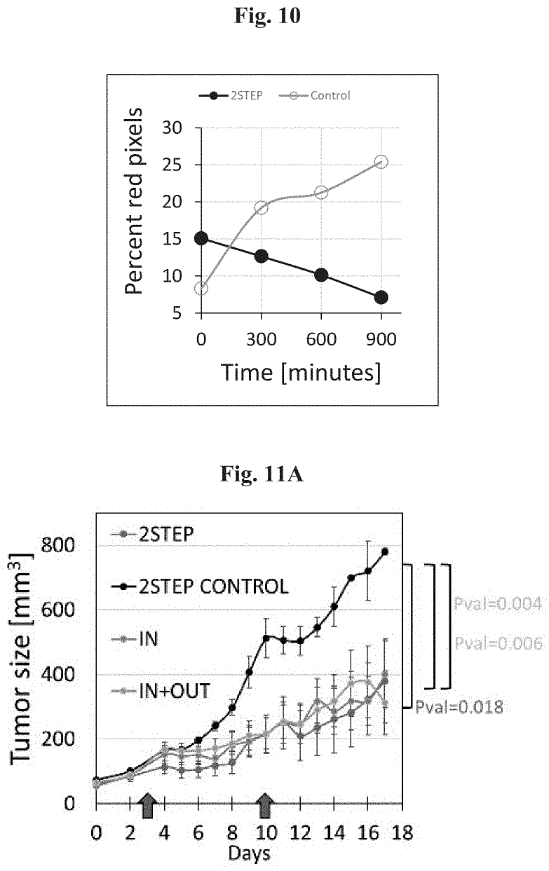

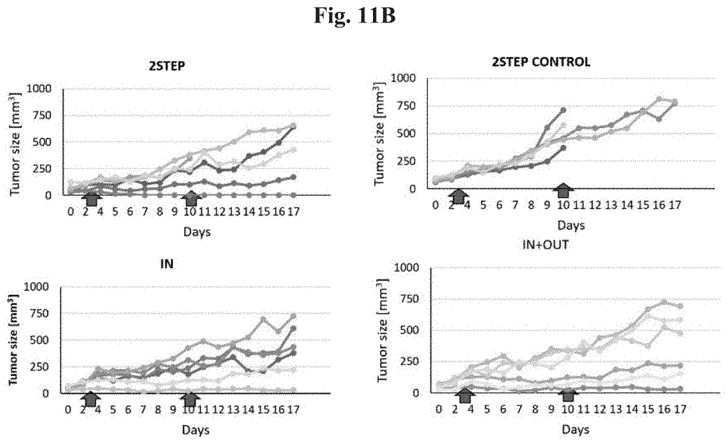

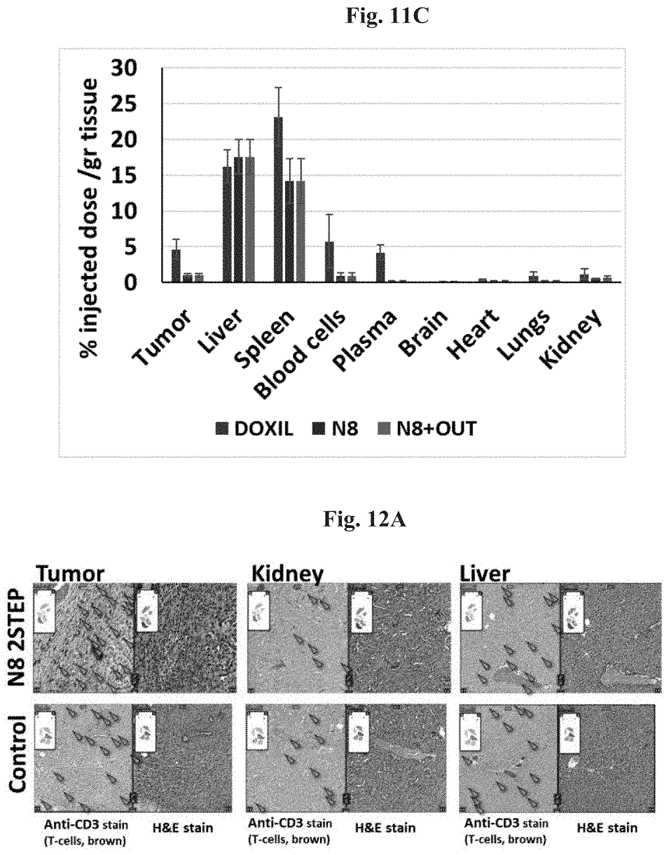

It has been found in accordance with the present invention that certain combinations of lipids with different proportions of positively charged (such as DOTAP) and zwitterion lipids (such as DAPE, diacyl phosphatidylethanolamine, DAPC) significantly improved the fusion with cancer cells. The liposomal labelling platform of the invention was used to preferentially label cancer cells with one functional group of a binding pair, such as click chemistry (FIG. 2A). This functional group is used to add an immune activating agent, such as monoclonal antibody (mAb) (Examples 1 to 4). It has further been shown herein that administration of the liposome of the present invention to animal models of cancer results in a bio-distribution profile that is similar to that of the DOXIL formulation (doxorubicin encapsulated in a liposome), which is routinely prescribed for treatment of cancer and is used as a benchmark or “Gold standard” for treatment (Example 5). In addition, administration of the liposome of the present invention carrying T cell activating antibodies results in significant T cell recruitment to tumors but not to liver and kidneys. Furthermore, it was found that liposomes incubated with primary tumor cells, kidney, liver lung or spleen cells and tested for fusion, underwent efficient fusion with the tumor cells, whereas healthy tissue-derived cells presented very low fusion with the immune labelling liposomes (FIG. 11B). Collectively, data shown in FIG. 10C, with FIGS. 11A-C, emphasize the selectivity of the liposomes towards tumor cells as opposed to healthy tissues such as liver in spite of their increased presence due to different modes of action. Finally, tumor growth was significantly inhibited in animals treated with the liposome of the present invention as compared with animals treated with a control liposome lacking the functional group of the T cell activating antibodies.

In one aspect, the present invention provides a supramolecular assembly comprising a plurality of lipids, wherein the hydrophilic head of at least one lipid of the supramolecular assembly is functionalised with a functional group or with one or more immune-system activating agents. This functional group is a member of a binding pair.

The term “binding pair” as used herein refers to a pair of different molecules, each comprising its own specific functional group, both functional groups have particular specificity for (or complimentary to) each other. In other words, these groups, under normal conditions, are capable of binding to each other in preference to binding to other molecules. The binding may be covalent or non-covalent. Non-limiting examples of such binding pairs are thiol-maleimide, azide-alkyne, aldehyde-hydroxylamine etc.

In general, a functional group is a specific group or moiety of atoms or bonds within molecules that is responsible for the characteristic chemical reactions of those molecules. In particular, a functional group, or a functional group of a binding pair, as defined herein, refers to a specific reactive group or moiety of atoms or bonds of the binding pair (hereinafter “a first functional group”) capable of binding to another functional group of said binding pair (hereinafter “a second functional group”). As mentioned above, the first and the second functional groups are complementary to each other. In the above non-limiting examples, the first functional groups are thiol, azide or aldehyde and their complementary (second) functional groups are maleimide, alkyne or hydroxylamine, respectively.

In general, crosslinking reagents (or crosslinkers) as defined herein refer to molecules that contain two or more reactive ends (functional groups) capable of chemically attaching to specific reactive groups (primary amines, sulfhydryls, etc.) on proteins or other molecules. In particular, the crosslinkers as defined herein comprise functional groups and spacers.

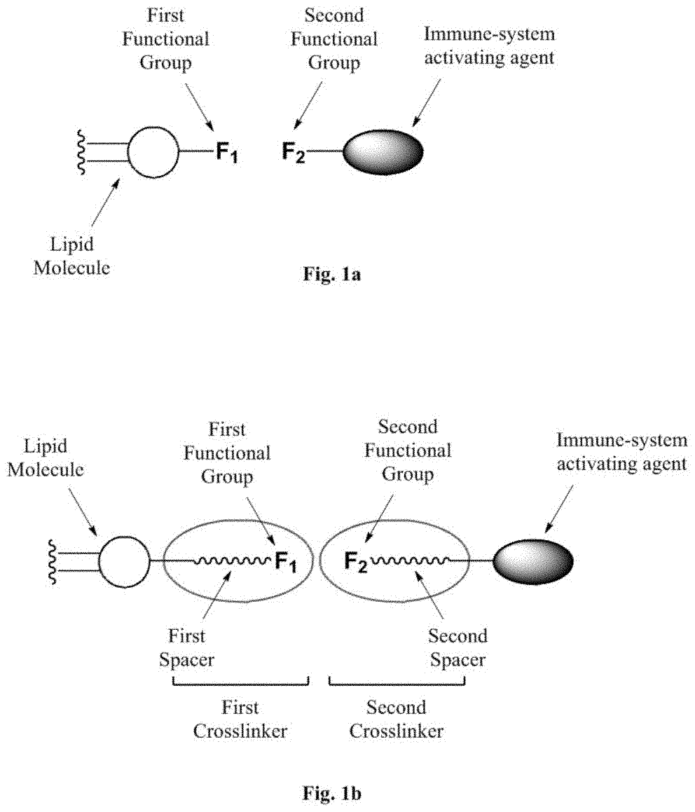

In certain embodiments, the first functional group, as defined herein, constitutes a reactive end of the first crosslinker, and the second functional group, as defined herein, constitutes a reactive end of the second crosslinker (FIG. 1B). In other embodiments, the spacers of the crosslinkers are omitted, thereby leaving only functional groups in the binding pair (FIG. 1A).

In certain embodiments, the supramolecular assembly is selected from a lipid particle, capable of fusing to or reacting with a target cell, such as a liposome, a micelle, and a cubosome; and a supramolecular assembly designed for releasing lipids within or in the vicinity of a tumor, such as a lipid gel, a lipid sponge, a bilayer or monolayer lipid sheet, a filamentous lipid structure or a lipid cochleate.

In one aspect, the present invention provides a method for treating cancer by labelling cancer cells with an immune-system activating agent, said method comprising administering to a cancer patient a fusogenic liposome, wherein the method comprises the steps of (i) administering to said cancer patient an immune-system activating fusogenic liposome comprising: (a) a lipid bilayer comprising a plurality of lipid molecules having 14 to 24 carbon atoms, and a first functional group of a specific binding pair capable of binding to a complementary second functional group of said binding pair; and (b) an immune-system activating agent comprising said complementary second functional group of said binding pair bound to said first functional group; or (ii) administering to said cancer patient a functionalised fusogenic liposome comprising a lipid bilayer comprising a plurality of lipid molecules having 14 to 24 carbon atoms, wherein at least one of said lipid molecules is functionalised with a first functional group of a specific binding pair capable of binding to a complementary second functional group of said binding pair; and subsequently to step (ii) administering an immune-system activating agent functionalised with a complementary second functional group of the binding pair capable of binding to said first functional group of said lipid molecules.

Similarly, the present invention provides (1) a fusogenic liposome for use in treating cancer by labelling cancer cells with an immune-system activating agent, wherein said fusogenic liposome comprises: (a) a lipid bilayer comprising a plurality of lipid molecules having 14 to 24 carbon atoms, and a first functional group of a specific binding pair capable of binding to a complementary second functional group of said binding pair; and (b) an immune-system activating agent comprising said complementary second functional group of said binding pair bound to said first functional group; or (2) a combination of a functionalised fusogenic liposome and a functionalised immune-system activating agent for use in treating cancer, wherein said functionalised fusogenic liposome comprises a lipid bilayer comprising a plurality of lipid molecules having 14 to 24 carbon atoms, wherein at least one of said lipid molecules is functionalised with a first functional group of a specific binding pair capable of binding to a complementary second functional group of said binding pair, and said functionalised immune-system activating agent is functionalised with a complementary second functional group of the binding pair capable of binding to said first functional group of said lipid molecules, and the combination is for administration by a dosage regimen comprising administration of said functionalised fusogenic liposome of prior to administration of said functionalised immune-system activating agent.

The term “liposome” as used herein refers to a lipid nanoparticle or construct comprising a lipid bilayer composed of an inner and an outer leaflet, which encapsulates an aqueous interior of the liposomes.

The term “fusogenic liposome” as used herein refers to a liposome construct that preferentially fuses with the plasma membrane of a target cell and is taken up by endocytosis to a lesser degree.

In general, as defined herein, the term “labelling (of) cells” relates to any modification of the cells structurally distinguishing them from the unmodified cells. In particular, the cells in the present invention are modified or “labeled” with a functional group of a fusogenic liposome or with an immune-system activating agent.

In one embodiment, said immune-system activating agent is bound via said second functional group to the first functional group of at least one of said lipid molecules at the outer leaflet of the fusogenic liposome.

In one embodiment, said immune-system activating agent is bound via said second functional group to the first functional group of at least one of said lipid molecules at the inner leaflet of the fusogenic liposome.

In one embodiment, said immune-system activating agent is bound via said second functional group to the first functional group of at least one of said lipid molecules at both the outer and inner leaflet of the fusogenic liposome.

In one embodiment, the immune-system activating agent is selected from a T-cell activating agent; a pro-inflammatory cytokine; a memory killer T cell activating peptide; soluble human leukocyte antigen (sHLA) presenting a viral peptide; and a super-antigen. In particular, the immune-system activating agent may be a T-cell activating agent, such as an anti-CD3 antibody, an anti-CD8 antibody, an anti-NKG2D antibody, or a combination thereof, an antibody capable of binding both CD3 and CD8 and an antibody capable of binding both CD3 and NKG2D, or an anti-NKG2D dimerizing antibody, or functional fragments of said antibodies (scFv or Fab); the pro-inflammatory cytokine is selected from IL2, IL-6, IL-17, IL-1, TNFα, and IFNγ, or a combination thereof, optionally reversibly linked to the lipid via a hydrolysable linker; the memory killer T cell activating peptide is an antimicrobial peptide such as an α-defensin; and the superantigen is staphylococcal toxic shock syndrome toxin-1, TSST-1 or similarly acting antigens that can bind T cell receptor to target cell's MHC/HLA and induce a cascade culminating in killer T cell activation.

The antibodies or functional fragments thereof described herein refer also to a single chain variable fragment (scFv); a functional fragment of an antibody; a single-domain antibody, such as a Nanobody; and a recombinant antibody; (ii) an antibody mimetic, such as an affibody molecule; an affilin; an affimer; an affitin; an alphabody; an anticalin; an avimer; a DARPin; a fynomer; a Kunitz domain peptide; and a monobody; or (iii) an aptamer.

It should be made clear that the antibodies or functional fragments thereof used in the present invention do not fulfill the function of targeting agent (to bring the liposome to a certain target cell), but instead fulfill the function of immune system activating agent.

In certain embodiments, the immune-activating agent may act by releasing immune repression exerted by immune checkpoints. The checkpoints that may be manipulated to release the immunosuppression in accordance with the present invention may be selected from the group consisting of PD1-PDL1, PD1-PDL2, CD28-CD80, CD28-CD86, CTLA4-CD80, CTLA4-CD86, ICOS-B7RP1, B7H3, B7H4, B7H7, B7-CD28-like molecule, BTLA-HVEM, KIR-MHC class I or II, LAG3-MHC class I or II, CD137-CD137L, OX40-OX40L, CD27-CD70, CD40L-CD40, TIM3-GAL9, V-domain Ig suppressor of T cell activation (VISTA), STimulator of INterferon Genes (STING), T cell immunoglobulin and immunoreceptor tyrosine-based inhibitory motif domain (TIGIT), A2aR-Adenosine and indoleamine-2,3-dioxygenase (IDO)-L-tryptophan.

Agents capable of blocking immune checkpoints are known in the art¹⁶ and these agents can be used in accordance with the present invention. Each one of the cited publications below, and Pardoll, 2012¹⁷, is incorporated by reference as if fully disclosed herein.

For example, anti-immune checkpoint antibodies, such as anti-PD1 antibodies, bound to liposomes could improve the half-life time of the antibodies. Alternatively, small molecule immune checkpoint inhibitors could be contained in liposomes and released in the tumor environment. Targeted release of such antibodies or small molecule inhibitors is expected to significantly reduce side-effects.

For example, the anti-PD-1 antibody used in accordance with the present invention may be selected from those disclosed in Ohaegbulam et al¹⁸, the entire contents of which being hereby incorporated herein by reference, i.e. CT-011 (pidilizumab; Humanized IgG1; Curetech), MK-3475 (lambrolizumab, pembrolizumab; Humanized IgG4; Merck), BMS-936558 (nivolumab; Human IgG4; Bristol-Myers Squibb), AMP-224 (PD-L2 IgG2a fusion protein; AstraZeneca), BMS-936559 (Human IgG4; Bristol-Myers Squibb), MEDI4736 (Humanized IgG; AstraZeneca), MPDL3280A (Human IgG; Genentech), MSB0010718C (Human IgG1; Merck-Serono); or the antibody used in accordance with the present invention may be MEDI0680 (AMP-514; AstraZeneca) a humanized IgG4 mAb.

The anti-CTLA4 antibody may be Tremelimumab (Pfizer), a fully human IgG2 monoclonal antibody; or ipilimumab, a fully human human IgG1 monoclonal antibody.

The anti-killer-cell immunoglobulin-like receptors (KIR) antibody may be Lirilumab (BMS-986015; developed by Innate Pharma and licenced to Bristol-Myers Squibb), a fully human monoclonal antibody.

The anti-LAG-3 antibody is directed against lymphocyte activation gene-3. One such antibody that may be used according to the present invention is the monoclonal antibody BMS-986016 (pembrolizumab; Humanized IgG4; Merck).

A representative list of small molecule immune checkpoint inhibitors is presented in Table 2.

TABLE 2

| | | | | Development | |
|---|---|---|---|---|---|
| Target | Inhibitor | Company/Institution | 1^{st} indications | Status | References |
| IDO | 1MT, MTH-Trp | NewFink | Oncology/HIV | Discovery | 15, 28 |
| ARG | BEC, ABH | University of Pennsylvania | Oncology | Discovery | 54 |
| iNOS | _{L}-NMMA | Fujisawa | Sepsis | Discontinued | 51, 133 |
| | PS-891169 | Pharmacopeia | Inflammation | Discovery | IDDB* |
| ARG/iNOS | NCX-4016 | NicOx SA | Oncology/Cardio | Phase II | 52 |
| COX2 | Celbrex | Pfizer | Inflammation | Faunched | 134 |
| | | | Oncology | Phase II | 63, 66 |
| | Rofecoxib | Merck | Inflammation/Oncology | Withdrawn | 62, 135 |
| EP2/EP4 | CP-533536 | Pfizer | Osteoporosis | Phase I | IDDB |
| TGFβRI | SB-505124 | Glaxo SmithKline | Inflammation/Oncology | Discovery | 136 |
| | SD-208 | J&J/Scios | Inflammation/Oncology | Discovery | 96 |
| | FY5 80276 | Filly | Inflammation/Oncology | Discovery | 137 |
| JAK/STAT | JSI-124 | H. Fee Moffitt Cancer Center | Oncology | Discovery | 101-103 |
| | CPA-7 | H. Fee Moffitt Cancer Center | Oncology | Discovery | 100, 138 |
| VEGF1(FLT1) | SU416 | Pfizer | Oncology | Discontinued | 116, 139 |
| | AG-13736 | Pfizer | Oncology | Phase II | IDDB |
| CCR4 | IC-487892 | ICOS | Inflammation | Discovery | IDDB |
| CXCR4 | AMD3100 | AnorMed | Oncology/HIV | Discovery | 140 |
| CCR2 | INCB3344 | Pfizer | Inflammation | Discovery | 141 |

*Investigational drugs database, ABH, 2(S)-amino-6-boronohexanoic acid; ARG, aginase, BEC, S-(2-boronoethyl)-_{L}-cysteine; COX2, cyclooxygenase 2; EP2, prostaglandin E receptor 2; FLT1, FMS-like tyrosine kinase 1; IDO, indoleamine 2,3-dioxygenase; iNOS, inducible nitric-oxide synthase; JAK Janus kinase; _{L}-NMMA, N^{C}-monomethyl-_{L}-arginine; 1MT, 1-methyl-trytophan; MTH-Trp, methylthiohydantoin-tryptophan; NCX-4016, nitroaspirin; STAT, signal transducer and activator of transcription; TGFβRI, transforming growth factor-β receptor type I; VEGFR1, vascular endothelial growth factor receptor 1.
Muller et al. Nature Reviews Cancer 6, 613-625 (August 2006)

In one embodiment, the liposome comprises a moiety that is cationic at physiological pH. Thus, in one embodiment, the at least one of said lipid molecules further comprises a cationic group, a cationic natural or synthetic polymer, a cationic amino sugar, a cationic polyamino acid or a cationic amphiphilic cancer-cell binding peptide.

In particular, at least one of said lipid molecules comprising a cationic group is selected from 1,2-dioleoyl-3-trimethylammoniumpropane chloride (DOTAP), dioctadecylamidoglycylspermine (DOGS), 1,2-di-O-octadecenyl-3-trimethylammonium propane (DOTMA), Dimethyldioctadecylammonium (18:0 DDAB), and N1-[2-((1S)-1-[(3-aminopropyl)amino]-4-[di(3-amino-propyl)amino]butyl-carboxamido)ethyl]-3,4-di[oleyloxy]-benzamide (MVL5); the synthetic polymer is selected from polyethyleneimines (PEI) and poly(2-(dimethylamino)ethyl methacrylate; the natural polymer is polysaccharide, such as chitosan; the amino sugar is glucosamine, the cationic polyamino acid is selected from poly(L-lysine), poly(L-arginine), poly(D-lysine), poly(D-arginine), poly(L-ornithine) and poly(D-ornithine); or said amphiphilic cancer-cell binding peptide is selected from Cecropin A (KWKLFKKIEKVGQNIRDGIIKAGPAVAVVGQATQIAK; (SEQ ID NO: 1); Cecropin A 1-8 (KWKLFKKI; (SEQ ID NO: 2) and cyclic CNGRC (SEQ ID NO: 3).

In a more particular embodiment, said lipid molecule comprising a cationic group is DOTAP.

In one embodiment, said at least one of said lipid molecules is a phospholipid selected from the group consisting of a phosphatidylcholine, a phosphatidylethanolamine, a phosphatidylserine, a phosphatidic acid or a combination thereof, each one of which comprises one or two identical or different fatty acid residues, wherein the fatty acid residues in the phosphatidyl moiety is saturated, mono-unsaturated or poly-unsaturated and has a carbon chain length of 14, 15, 16, 17, 18, 19, 20, 21, 22, 23 or 24 carbons, such as myristoyl, stearoyl, palmitoyl, oleoyl, linoleoyl, linolenoyl (including conjugated linolenoyl), arachidonoyl in phospholipid and lyso-phospholipid configuration, and combinations thereof.

In particular embodiments, said phospholipid is selected from the group consisting of 1-palmitoyl-2-oleoyl-sn-glycero-3-phosphocholine (POPC) and 1,2-dioleoyl-3-phosphatidylethanolamine (DOPE); 1,2-dimyristoyl-3-phosphatidylcholine (DMPC); 1,2-distearoyl-3-phosphatidylcholine (DSPC); 1,2-dimyristoleoyl-sn-glycero-3-phosphocholine (14:1 (Δ9-Cis) PC); 1,2-dimyristelaidoyl-sn-glycero-3-phosphocholine (14:1 (Δ9-Trans) PC); 1,2-dipalmitoleoyl-sn-glycero-3-phosphocholine (16:1 (Δ9-Cis) PC); 1,2-dipalmitelaidoyl-sn-glycero-3-phosphocholine (16:1 (Δ9-Trans) PC); 1,2-dipetroselenoyl-sn-glycero-3-phosphocholine (18:1 (Δ6-Cis) PC); 1,2-dioleoyl-3-phosphatidylcholine (18:1 (49-Cis) PC (DOPC)); 1,2-dielaidoyl-sn-glycero-3-phosphocholine (18:1 (Δ9-Trans) PC); 1,2-dilinoleoyl-sn-glycero-3-phosphocholine (18:2 (Cis) PC (DLPC)); 1,2-dilinolenoyl-sn-glycero-3-phosphocholine (18:3 (Cis) PC); 1,2-dieicosenoyl-sn-glycero-3-phosphocholine (20:1 (Cis) PC); 1,2-diarachidonoyl-sn-glycero-3-phosphocholine (20:4 (Cis) PC); 1,2-didocosahexaenoyl-sn-glycero-3-phosphocholine (22:6 (Cis) PC); 1,2-dierucoyl-sn-glycero-3-phosphocholine (22:1 (Cis) PC); 1,2-dinervonoyl-sn-glycero-3-phosphocholine (24:1 (Cis) PC); 1,2-dimyristoyl-3-3-phosphatidylethanolamine (DMPE); 1,2-dipalmitoyl-3-phosphatidylethanolamine (DPPE); dipalmitoylphosphatidylcholine (DPPC); 1,2-dioleoyl-3-phosphatidylethanolamine (DOPE); 1,2-distearoyl-3-phosphatidylethanolamine (DSPE); 1,2-dimyristoyl-3-phosphatidylserine (DMPS); 1,2-dipalmitoyl-3-phosphatidylserine (DPP S); palmitoyloleoyl phosphatidylethanolamine (POPE); and 1,2-dioleoyl-3-phosphatidylserine (DOPS). More particularly, said phospholipid is selected from DOPC, POPC, DMPC, DPPC, DOPE, POPE, DSPE, DMPE and DPPE.

In one embodiment, the fusogenic liposome further comprises a stabilizing moiety connected to at least one of said lipid molecules.

The term “stabilizing moiety” as used herein refers to a moiety that when incorporated within the lipid bilayer of the liposome provides prolonged blood circulation half-life of the liposomes as compared with an identical liposome lacking the stabilizing moiety.

In a particular embodiment, said stabilizing moiety is selected from polyethylene glycol (PEG), polypropylene glycol, polyvinyl alcohol, polyvinylpyrrolidone (PVP), dextran, a polyamino acid, methyl-polyoxazoline, polyglycerol, poly(acryloyl morpholine), and polyacrylamide. For instance, the stabilizing moiety is PEG of molecular weight of about 106 Da to about 4 kDa, for example: 106 Da (PEG₂), 194 Da (PEG₄), 600 Da (PEG600), 2 kDa (PEG2000), and 4 kDa (PEG4000). In more particular embodiment, the stabilizing moiety is PEG of molecular weight of about 2 kDa.

In one embodiment, said stabilizing moiety is connected to at least one of said lipid molecules via a cleavable peptide linker, such as VPMSMRGG (SEQ ID NO: 4) for matrix metalloproteinase (MMP)-1, IPVSLRSG (SEQ ID NO: 5) or GGGGPLGVRGGGGK (SEQ ID NO: 6) for MMP-2, RPFSMIMG (SEQ ID NO: 7) for MMP-3, VPLSLTMG (SEQ ID NO: 8) for MMP-7, VPLSLYSG (SEQ ID NO: 9) for MMP-9 and IPESLRAG (SEQ ID NO: 10) for membrane type 1-matrix metalloproteinase (MT1-MMP), all of which can be modified at the N and/or C terminus with amino acid residues, PEGs and other linkers.

In certain embodiments, the cleavable linker is a pH-sensitive cleavable linker such as dithiodipropionateaminoethanol (DTP) or dithio-3-hexanol (DTH).

In certain embodiments, the supramolecular assembly designed for releasing lipids, but not the fusogenic liposome, comprises a polymer, such as PEG, poly(lactic-co-glycolic acid) (PLGA), and alginate.

In certain embodiments, the hydrophilic head of the at least one lipid of the plurality of lipids is each functionalised with a first functional group or a second functional group of a binding pair capable of binding to each other under normal conditions in preference to binding to other molecules or forming between themselves a covalent bond or non-covalent high-affinity conjugate, wherein the first functional group and the second functional group of the binding pair is for example, but is not limited to, (i) reactive groups of a click chemistry reaction; or (ii) a biotin and a biotin-binding peptide or biotin-binding protein.

The term “high affinity” as used herein refers to a chemical or bio-physical association, such as chelator-metal coupling (e.g. Ni and a peptide sequence comprising several His-residues such as His6), or an conjugation between two members of a binding pair, e.g. an antibody and its target epitope or biotin and streptavidin, etc., wherein the association between two binding pairs has a K_{d} of 10⁻⁴ M to 10⁻³⁰ M, e.g. 10⁻⁶ M, 10⁻⁷ M, 10⁻⁸ M, 10⁻⁹ M, 10⁻¹⁰ M, 10⁻¹¹ M, 10⁻¹² M or 12⁻¹³ M.

In one embodiment, said first functional group of the specific binding pair is capable of forming a covalent bond with said complementary second functional group of said binding pair.

In a particular embodiment, said first functional group of the specific binding pair is capable of forming a covalent bond with said complementary second functional group of said binding pair via a click chemistry reaction.

In a particular embodiment, i) the first functional group of the specific binding pair is alkyne or phosphine, and the second functional group of said binding pair is azide, or vice versa; ii) the first functional group of the specific binding pair is cycloalkene, cycloalkyne, cyclopropane, isonitrile (isocyanide) or vinyl boronic acid, and the second functional group of said binding pair is tetrazine, or vice versa; iii) the first functional group of the specific binding pair is alkyne or maleimide, and the second functional group of said binding pair is thiol, or vice versa; iv) the first functional group of the specific binding pair is conjugated diene, and the second functional group of said binding pair is substituted alkene, or vice versa; v) the first functional group of the specific binding pair is alkene, alkyne or copper acetylide, and the second functional group of said binding pair is nitrone, or vice versa; vi) the first functional group of the specific binding pair is aldehyde or ketone, and the second functional group of said binding pair is alkoxyamine, hydroxylamine, hydrazine or hydrazide, or vice versa; or vii) the first functional group of the specific binding pair is aldehyde, ketone, isothiocyanate, carboxylic acid or derivative thereof such as ester, anhydride, acyl halide, tosyl and N-hydrosuccinimide (NHS), and the second functional group of said binding pair is amine, or vice versa. In a more particular embodiment, the specific binding pair is alkyne-azide.

In one embodiment, said first functional group of the specific binding pair is capable of forming a non-covalent bond with said complementary second functional group of said binding pair.

In a particular embodiment, the first functional group of the specific binding pair is biotin, and the second functional group of said binding pair is its binding-partner selected from a biotin-binding peptide or biotin-binding protein, or vice versa. For example, said biotin-binding protein may be selected from avidin, streptavidin and an anti-biotin antibody; and said biotin-binding peptide is selected from AEGEFCSWAPPKASCGDPAK (SEQ ID NO: 11), CSWRPPFRAVC (SEQ ID NO: 12), CSWAPPFKASC (SEQ ID NO: 13), and CNWTPPFKTRC (SEQ ID NO: 14) [Saggio and Laufer. Biotin binders selected from a random peptide library expressed on phage. Biochem. J. (1993) 293, 613-616; herein incorporated by reference as if fully enclosed]. The Cysteine residues may form a disulfide bond and linkers could be attached to the N- or C-terminus or both termini.

In one embodiment, the fusogenic liposome further comprises a first spacer between the lipid bilayer and the first functional group (FIG. 1B).

In one embodiment, the immune-system activating agent further comprises a second spacer between the immune-system activating agent and the second functional group (FIG. 1B).

In one embodiment, the first or second spacer is selected from the group consisting of PEG, (C₆-C₁₂)alkyl, phenolic, benzoic or naphthoic mono-, di- or tricarboxylic acid, tetrahydropyrene mono-, di- or tri-carboxylic acid, or salts thereof, cyclic ether, glutaric acid, succinate acid, muconic acid, adipic acid, pimelic acid, suberic acid, azelaic acid, and sebacic acid, and a peptide, such as a poly-Gly peptide of about 2-20 amino acid residues in length, e.g. 3 amino acid residues in length.

In a particular embodiment, the first or second spacer is PEG of molecular weight of about 106 Da to about 4 kDa, for example: 106 Da (PEG₂), 194 Da (PEG₄), 600 Da (PEG600), 2 kDa (PEG2000), and 4 kDa (PEG4000); and more particularly the first or second spacer is PEG of a molecular weight of about 194 Da (PEG₄).

Alternatively, in a particular embodiment, the first or second spacer is (C₆-C₁₂)alkyl, preferably heptyl or dodecanoyl.

In one embodiment, the fusogenic liposome further comprises cholesterol (CHO) or its derivatives.

In one embodiment, the liposome has a size up to 200 nm, e.g. from about 15 nm to about 200 nm, from about 20 nm to about 100 nm, from about 50 nm to about 150 nm, from about 50 nm to about 90 nm, from about 80 nm to about 100 nm, from about 110 nm to about 200 nm, e.g. about 100 nm.

In certain embodiments, the fusogenic liposome further comprises in its hydrophilic core one or more immune-system activating agents such as a pro-inflammatory cytokine, e.g. IL2, IL-6, IL-17, IL-1, TNFα, and IFNγ; at least one stimulating molecule, e.g. ionomycin; and at least one memory killer T cell activating peptide.

In certain embodiments, said immune-system activating agent is bound via said second functional group to the first functional group of at least one of said lipid molecules at the outer leaflet, inner leaflet, or both outer and inner leaflet of the fusogenic liposome; the immune-system activating agent is selected from a T-cell activating agent; a pro-inflammatory cytokine; a memory killer T cell activating peptide; and a super-antigen; at least some of said lipids further comprise a cationic group, a cationic natural or synthetic polymer, a cationic amino sugar, a cationic polyamino acid or an amphiphilic cancer-cell binding peptide; at least some of the lipids are phospholipids selected from the group consisting of a phosphatidylcholine, a phosphatidylethanolamine, a phosphatidylserine, a phosphatidic acid or a combination thereof, each one of which comprises one or two identical or different fatty acid residues, wherein the fatty acid residues in the phosphatidyl moiety is saturated, mono-unsaturated or poly-unsaturated and has a carbon chain length of 14, 15, 16, 17, 18, 19, 20, 21, 22, 23 or 24 carbons, such as myristoyl, stearoyl, palmitoyl, oleoyl, linoleoyl, linolenoyl (including conjugated linolenoyl), arachidonoyl in phospholipid and lyso-phospholipid configuration, and combinations thereof; said liposome further comprises a stabilizing moiety connected to at least one of said lipids or said cationic polymers; said first functional group of the specific binding pair is capable of forming a covalent bond with said complementary second functional group of said binding pair or said first functional group of the specific binding pair is capable of forming a non-covalent bond with said complementary second functional group of said binding pair; the first or second spacer is selected from the group consisting of PEG, (C₆-C₁₂)alkyl, phenolic, benzoic or naphthoic mono-, di- or tricarboxylic acid, tetrahydropyrene mono-, di- or tri-carboxylic acid, or salts thereof, cyclic ether, glutaric acid, succinate acid, muconic acid, adipic acid, pimelic acid, suberic acid, azelaic acid, and sebacic acid, a peptide, such as a poly-Gly peptide of about 2-20 amino acid residues in length, e.g. 3 amino acid residues in length; and the liposome has a size up to 200 nm, e.g. from about 15 nm to about 200 nm, from about 20 nm to about 100 nm, from about 50 nm to about 150 nm, from about 50 nm to about 90 nm, from about 80 nm to about 100 nm, from about 110 nm to about 200 nm, e.g. about 100 nm.

In a particular embodiment, the immune-system activating agent is a T-cell activating agent; said at least one of said lipid molecules comprising a cationic group is selected from 1,2-dioleoyl-3-trimethylammoniumpropane chloride (DOTAP), dioctadecylamidoglycylspermine (DOGS), 1,2-di-O-octadecenyl-3-trimethylammonium propane (DOTMA), Dimethyldioctadecylammonium (18:0 DDAB), and N1-[2-((1 S)-1-[(3-aminopropyl)amino]-4-[di(3-amino-propyl)amino]butyl-carboxamido)ethyl]-3,4-di[oleyloxy]-benzamide (MVL5), said synthetic polymer is selected from polyethyleneimines (PEI) and poly(2-(dimethylamino)ethyl methacrylate, said natural polymer is chitosan, said amino sugar is glucosamine; said cationic polyamino acid is selected from poly(L-lysine), poly(L-arginine), poly(D-lysine), poly(D-arginine), poly(L-ornithine) and poly(D-ornithine), or said amphiphilic cancer-cell binding peptide is selected from Cecropin A; Cecropin A 1-8; and cyclic CNGRC; said at least one of said lipid molecules is selected from the group consisting of 1-palmitoyl-2-oleoyl-sn-glycero-3-phosphocholine (POPC) and 1,2-dioleoyl-3-phosphatidylethanolamine (DOPE); 1,2-dimyristoyl-3-phosphatidylcholine (DMPC); 1,2-di stearoyl-3-phosphatidylcholine (DSPC); 1,2-dimyristoleoyl-sn-glycero-3-phosphocholine (14:1 (Δ9-Cis) PC); 1,2-dimyristelaidoyl-sn-glycero-3-phosphocholine (14:1 (Δ9-Trans) PC); 1,2-dipalmitoleoyl-sn-glycero-3-phosphocholine (16:1 (Δ9-Cis) PC); 1,2-dipalmitelaidoyl-sn-glycero-3-phosphocholine (16:1 (Δ9-Trans) PC); 1,2-dipetroselenoyl-sn-glycero-3-phosphocholine (18:1 (Δ6-Cis) PC); 1,2-dioleoyl-3-phosphatidylcholine (18:1 (Δ9-Cis) PC (DOPC)); 1,2-dielaidoyl-sn-glycero-3-phosphocholine (18:1 (Δ9-Trans) PC); 1,2-dilinoleoyl-sn-glycero-3-phosphocholine (18:2 (Cis) PC (DLPC)); 1,2-dilinolenoyl-sn-glycero-3-phosphocholine (18:3 (Cis) PC); 1,2-dieicosenoyl-sn-glycero-3-phosphocholine (20:1 (Cis) PC); 1,2-diarachidonoyl-sn-glycero-3-phosphocholine (20:4 (Cis) PC); 1,2-didocosahexaenoyl-sn-glycero-3-phosphocholine (22:6 (Cis) PC); 1,2-dierucoyl-sn-glycero-3-phosphocholine (22:1 (Cis) PC); 1,2-dinervonoyl-sn-glycero-3-phosphocholine (24:1 (Cis) PC); 1,2-dimyristoyl-3-3-phosphatidylethanolamine (DMPE); 1,2-dipalmitoyl-3-phosphatidylethanolamine (DPPE); dipalmitoylphosphatidylcholine (DPPC); 1,2-dioleoyl-3-phosphatidylethanolamine (DOPE); 1,2-di stearoyl-3-phosphatidylethanolamine (D SPE); 1,2-dimyristoyl-3-phosphatidylserine (DMPS); 1,2-dipalmitoyl-3-phosphatidylserine (DPP S); palmitoyloleoyl phosphatidylethanolamine (POPE); and 1,2-dioleoyl-3-phosphatidylserine (DOPS); said stabilizing moiety is selected from polyethylene glycol (PEG), polypropylene glycol, polyvinyl alcohol, polyvinylpyrrolidone (PVP), dextran, a polyamino acid, methyl-polyoxazoline, polyglycerol, poly(acryloyl morpholine), and polyacrylamide; said first functional group of the specific binding pair is capable of forming a covalent bond with said complementary second functional group of said binding pair via a click chemistry reaction, i) the first functional group of the specific binding pair is alkyne or phosphine, and the second functional group of said binding pair is azide, or vice versa; ii) the first functional group of the specific binding pair is cycloalkene, cycloalkyne, cyclopropane, isonitrile (isocyanide) or vinyl boronic acid, and the second functional group of said binding pair is tetrazine, or vice versa; iii) the first functional group of the specific binding pair is alkyne or maleimide, and the second functional group of said binding pair is thiol, or vice versa; iv) the first functional group of the specific binding pair is conjugated diene, and the second functional group of said binding pair is substituted alkene, or vice versa; v) the first functional group of the specific binding pair is alkene, alkyne or copper acetylide, and the second functional group of said binding pair is nitrone, or vice versa; vi) the first functional group of the specific binding pair is aldehyde or ketone, and the second functional group of said binding pair is alkoxyamine, hydroxylamine, hydrazine or hydrazide, or vice versa; or vii) the first functional group of the specific binding pair is aldehyde, ketone, isothiocyanate, carboxylic acid or derivative thereof such as ester, anhydride, acyl halide, tosyl and N-hydrosuccinimide (NHS), and the second functional group of said binding pair is amine, or vice versa; viii) functional group, or the first functional group of the specific binding pair is biotin, and the second functional group of said binding pair is its binding-partner selected from a biotin-binding peptide or biotin-binding protein, or vice versa; and the first or second spacer is PEG of molecular weight of about 106 Da to about 4 kDa, or (C₆-C₁₂)alkyl, preferably heptyl or dodecanoyl.

In a more particular embodiment, the T-cell activating agent is selected from an anti-CD3 antibody, an anti-CD8 antibody, an anti-NKG2D antibody, or a combination thereof, an antibody capable of binding both CD3 and CD8 and an antibody capable of binding both CD3 and NKG2D, or an anti-NKG2D dimerizing antibody; said at least one of said lipid molecules comprising a cationic group is DOTAP; said phospholipid is selected from DOPC, POPC, DMPC, DPPC, DOPE, POPE, DSPE, DMPE and DPPE; the stabilizing moiety is PEG of molecular weight of about 106 Da to about 4 kDa; the specific binding pair is alkyne-azide, said biotin-binding protein is selected from avidin, streptavidin and an anti-biotin antibody, or said biotin-binding peptide is selected from AEGEFCSWAPPKASCGDPAK (SEQ ID NO: 11), CSWRPPFRAVC (SEQ ID NO: 12), CSWAPPFKASC (SEQ ID NO: 13), and CNWTPPFKTRC (SEQ ID NO: 14); and the first or second spacer is PEG of a molecular weight of about 194 Da (PEG₄).

In a more particular embodiment, the stabilizing moiety is PEG of molecular weight of about 2 kDa.

In a first certain particular embodiments, the fusogenic liposome comprises (a) DOPC:DOTAP:DSPE-PEG2K:DOPE-PEG4-N3 or DOPC:DOTAP:DSPE-PEG2K:DOPE-PEG4-BCN; or (b) DMPC:Cholesterol:DMPE-PEG4-N3 or DMPC:Cholesterol:DMPE-PEG4-BCN, wherein PEG2K represents PEG having a molecular weight of about 2 kDa and PEG4 represents PEG having a molecular weight of about 194 Da, and the relative molar amount of DOPC is up to about 80%, the relative molar amount of DOTAP is up to about 80%, the relative molar amount of DSPE-PEG2K is up to about 20%, the relative molar amount of DOPE-PEG4 is up to about 20%, the relative molar amount of HSPC is up to about 65%, the relative molar amount of Cholesterol is up to about 40%, and the relative molar amount of DMPC is up to about 70%, and the fusogenic liposome has a size of about 50 nm to about 300 nm, e.g. 80 nm or 100 nm.

In a second certain particular embodiments, the fusogenic liposome comprises (i) DOPC:DOTAP:DSPE-PEG2K:DOPE-PEG₄-N₃ or DOPC:DOTAP:DSPE-PEG2K:DOPE-PEG₄-BCN, in the molar ratio 52.5:35:0.6:10, 52.5:35:1.25:10, 52.5:35:2.5:10, 52.5:35:5:10, 52.5:35:0.6:5, 52.5:35:1.25:5, 52.5:35:2.5:5, 52.5:35:5:5, 65:20:5:10, 50:35:5:10, 52.5:35:1.25:7, 52.5:35:1.25:5, or 52.5:35:2.5:7: or (ii) DMPC:Cholesterol:DMPE-PEG₄-N₃ or DMPC:Cholesterol:DMPE-PEG₄-BCN, in the molar ratio 60:35:5.

In a third certain particular embodiments, the fusogenic liposome comprises DOPC:DOTAP:DSPE-PEG2K:DOPE-PEG₄-N₃ in the molar ratio 52.5:35:2.5:5 or 52.5:35:2.5:10.

In the first to third certain particular embodiments, said T-cell activating agent is conjugated via said second crosslinker to the first crosslinker of at least one of said lipid molecules at the outer leaflet of the fusogenic liposome.

In the first to third certain particular embodiments, said T-cell activating agent is conjugated via said second crosslinker to the first crosslinker of at least one of said lipid molecules at the inner leaflet of the fusogenic liposome

In the first to third certain particular embodiments, said T-cell activating agent is conjugated via said second crosslinker to the first crosslinker of at least one of said lipid molecules at both the inner and outer leaflet of the fusogenic liposome.

In the first to third certain particular embodiments, said T-cell activating agent is conjugated via said second functional group to the first functional group of at least one of said lipid molecules at the outer leaflet of the fusogenic liposome.

In the first to third certain particular embodiments, said T-cell activating agent is conjugated via said second functional group to the first functional group of at least one of said lipid molecules at the inner leaflet of the fusogenic liposome In the first to third certain particular embodiments, said T-cell activating agent is conjugated via said second functional group to the first functional group of at least one of said lipid molecules at both the inner and outer leaflet of the fusogenic liposome.

In certain embodiments, the first step of (ii) is performed immediately, 1 hr, 2 hr, 3 hr, 4 hr, 5 hr, 6 hr, 12 hrs, 1 day, 2 days, 3 days or up to 1 week before the second step of (iii).

In certain embodiments, the melting temperature (Tm) of the liposome of any one of the above recited embodiments is below 45° C., at which the fusogenic liposome is maintained at a non-crystalline transition phase thereby providing membrane fluidity required for fusion of liposome with cell membranes.

In certain embodiments, the cancer being treated using the method of any one of the above recited embodiments, is selected from the group consisting of breast cancer, such as triple-negative breast cancer, melanoma and lung cancer.

In another aspect, the present invention provides a fusogenic liposome comprising a lipid bilayer comprising a plurality of lipid molecules having 14 to 24 carbon atoms, wherein at least one of said lipid molecules is functionalised with a first functional group of a specific binding pair capable of binding to a complementary second functional group of said binding pair.

The components and size of the fusogenic liposome, such as the lipid molecules, functional groups, spacers, immune system activating agent, cationic group, cationic natural or synthetic polymer, cationic amino sugar, cationic polyamino acid or amphiphilic cancer-cell binding peptide, and stabilizing moiety, are as defined in the embodiments above relating to the method of treatment in which they may be used.

In certain embodiments, the fusogenic liposome further comprises a first spacer between the lipid bilayer and the first functional group.

In certain embodiments, the fusogenic liposome further comprises an immune system activating agent functionalised with a complementary second functional group of said binding pair bound to said first functional group.

In certain embodiments, the immune-system activating agent is bound via said second functional group to the first functional group of at least one of said lipid molecules at the outer leaflet of the fusogenic liposome.

In certain embodiments, the immune-system activating agent is bound via said second functional group to the first functional group of at least one of said lipid molecules at the inner leaflet of the fusogenic liposome.

In certain embodiments, the immune-system activating agent is bound via said second functional group to the first functional group of at least one of said lipid molecules at both the outer and inner leaflet of the fusogenic liposome.

In certain embodiments, the immune-system activating agent further comprises a second spacer between the immune-system activating agent and the second functional group.

In certain embodiments, the immune-system activating agent is selected from a T-cell activating agent; a pro-inflammatory cytokine; a memory killer T cell activating peptide; and a super-antigen.

In certain embodiments, the immune-system activating agent is a T-cell activating agent.

In certain embodiments, the T-cell activating agent is selected from an anti-CD3 antibody, an anti-CD8 antibody, or a combination thereof; and an antibody capable of binding both CD3 and CD8.

In an additional aspect, the present invention provides method for preparation of a fusogenic liposome with an immune system activating agent bound at the outer leaflet, said method comprising the reaction of a functionalised fusogenic liposome comprising (a) a lipid bilayer comprising a plurality of lipid molecules having 14 to 24 carbon atoms and a first functional group of a specific binding pair capable of binding to a complementary second functional group of said binding pair with an immune system activating agent functionalised with a complementary second functional group of the binding pair, wherein said second functional group binds to said first functional group, thereby yielding said fusogenic liposome conjugated to said T-cell activating agent bound at the outer leaflet.

In yet an additional aspect, the present invention provides a method for preparation of a fusogenic liposome with an immune system activating agent bound at both the inner and outer leaflet, said method comprising the steps of (i) reacting a plurality of lipid molecules having 14 to 24 carbon atoms, wherein at least one of said lipid molecules is functionalised with a first functional group of a specific binding pair, with a T-cell activating agent functionalised with a second functional group of the binding pair, wherein said second functional group binds to said first functional group of said lipid molecules, thereby yielding the lipid molecules linked to the T-cell activating agent; and (ii) preparing said fusogenic liposome from said lipid molecules obtained in step (i), thereby yielding the fusogenic liposome functionalised with said T-cell activating agent bound at both the inner and outer leaflet.

In still an additional aspect, the present invention provides a method for preparation of a fusogenic liposome with an immune system activating agent bound at the inner leaflet. The method is based on the concept of a kinetic reaction control. The liposomes are self-assembled from lipid bilayers at much higher reaction rate than the chemical bond is formed between two functional groups. Thus, an unreacted immune system activating agent and other reagents or catalysts, such as copper catalyst for the copper-dependent click-chemistry reaction, are encapsulated within the aqueous interior of the liposome before any significant chemical reaction occurs in the solution. The immune system activating agent and/or other reagents needed for the chemical reaction are not encapsulated inside the liposome are further physically removed from the solution, for example by washing the formed liposomes. Alternatively, the reaction conditions, such as pH of the solution, may be changed at some point to stop or inhibit the chemical reaction occurring outside the liposome, while the reaction conditions within the aqueous interior of the liposome remain unchanged due to the lipid bilayer barrier. Non-limiting examples of catalysts for the click chemical reaction to form the liposomes of the present invention are copper (II) acetylacetonate, copper (I) isonitrile and any other active copper (I) catalyst generated from copper (I) salts or copper (II) salts using sodium ascorbate as the reducing agent. The immune system activating agent and other reagents or catalysts may be removed by e.g. dialysis or gel filtration or by reacting one or both of the functional groups of the immune activating agent or lipids with an excess of a corresponding free functional group which depletes the functional groups of the immune activating agent or lipids and thus, stops or inhibits the reaction.

Thus, the method for preparation of a fusogenic liposome with an immune system activating agent bound at the inner leaflet comprises the following steps. In the first step, a plurality of lipid molecules having 14 to 24 carbon atoms, wherein at least one of said lipid molecules is functionalised with a first functional group of a specific binding pair, are mixed in a solution with a T-cell activating agent functionalised with a second functional group of the binding pair capable of binding at suitable reaction conditions to said first functional group of said lipid molecules. Without introducing any reagents or catalysts, the chemical reaction between the two functional groups is relatively slow. As a result, the lipid molecules are self-assembled into liposomes in the first reaction step, thereby encapsulating some portion of said T-cell activating agent molecules within the aqueous interior of the liposomes. In the second step, the T-cell activating agent molecules, which have not been encapsulated and remained in the solution outside the liposomes are removed or washed away. Optionally, the reaction of the lipid molecules with the non-encapsulated T-cell activating agent molecules may be inhibited as described above. In the third step, the reaction of the lipid molecules with the encapsulated T-cell activating agent inside the aqueous interior of the liposomes prepared in first step is carried out, wherein said second functional group of said T-cell activating agent binds to said first functional group of said lipid molecules, thereby yielding the fusogenic liposome functionalised with said T-cell activating agent bound at the inner leaflet.

In certain embodiments, the method for preparation of a fusogenic liposome with an immune system activating agent bound at the inner leaflet comprises the steps of (i) preparation of liposomes in a solution comprising a plurality of lipid molecules having 14 to 24 carbon atoms, wherein at least one of said lipid molecules is functionalised with a first functional group of a specific binding pair, and a T-cell activating agent functionalised with a second functional group of the binding pair capable of binding to said first functional group of said lipid molecules, thereby encapsulating a fraction of said T-cell activating agent; (ii) removal of non-encapsulated T-cell activating agent from the solution and all optional reagents and catalysts; (iii) reaction of the lipid molecules with the encapsulated T-cell activating agent inside the aqueous interior of the liposomes prepared in step (i), wherein said second functional group of said T-cell activating agent binds to said first functional group of said lipid molecules, thereby yielding the fusogenic liposome functionalised with said T-cell activating agent bound at the inner leaflet.

In certain embodiments, the solution further comprises at least one oxidation-reduction catalyst. In particular embodiments, the at least one oxidation-reduction catalyst is a copper (I) salt, which is removed in step (ii) in addition to the non-encapsulated T-cell activating agent, and the reaction in step (iii) is a copper-dependent click chemistry reaction.

Methods of preparing liposomes are well known in the art¹⁹. For example, a lipid solution in an organic solvent may be injected into an aqueous solution having a temperature above the Tm at conditions leading to formation of liposomes e.g. by the means of a nano-assembler assembler or other similar devices, thereby producing fusogenic liposomes; or injecting the lipid solution into an aqueous solution having a temperature above the Tm and mixing, thereby obtaining a liposome solution, and extruding the liposome solution through an extruder comprising at least one support and at least one etched membrane having pores with a diameter between 50 and 400 nm.

In a further aspect, the present invention provides a kit comprising (a) a first container comprising a fusogenic liposome comprising (a) a lipid bilayer comprising a plurality of lipid molecules having 14 to 24 carbon atoms, wherein at least one of said lipid molecules is functionalised with a first functional group of a specific binding pair capable of binding to a complementary second functional group of said binding pair; (b) a second container comprising a T-cell activating agent functionalised with a second functional group of the binding pair capable of binding to said first functional group of said lipid molecules; and (c) a pamphlet with instructions for a method for treating cancer comprising administering to a cancer patient the fusogenic liposome of (a) and subsequently the T-cell activating agent of (b).

In certain embodiments, the supramolecular assembly comprises dioleoylphosphatidylethanolamine (DOPE), optionally cholesterylhemisuccinate (CHEMS) and optionally distearoylphosphatidylethanolamine (DSPE) linked to methoxy-PEG (mPEG) via dithiodipropionateaminoethanol (DTP) or 1,2-Distearoyl-sn-glycero-3-phosphatidic acid (DSPA) linked to mPEG via dithio-3-hexanol (DTH), wherein the supramolecular assembly is destabilized at acidic pH, i.e. undergo acid-triggered destabilization. The pH-sensitive formulation may have a molar ratio of DOPE:CHEMS of 6:4 and 5-15% of mPEG-DTP-DSPE or mPEG-DTH-DSPA.

In yet an additional aspect, the present invention provides a pharmaceutical composition comprising the fusogenic liposome as defined in any one of the above embodiments and a pharmaceutically acceptable carrier.

In certain embodiments, the fusogenic liposome of any one of the above embodiments lacks a targeting agent.

The term “carrier” refers to a diluent, adjuvant, excipient, or vehicle with which the active agent is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

The compositions may be formulated for parenteral administration by injection, e.g., by bolus injection or continuous infusion or direct-tumor injection. Formulations for injection may be presented in unit dosage form, e.g., in ampoules or in multidose containers, with an added preservative or stabilizer. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

For oral administration, the pharmaceutical preparation may be in liquid form, for example, solutions, syrups or suspensions, or may be presented as a drug product for reconstitution with water, injectable isotonic, or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Preparations for oral administration may be suitably formulated to give controlled release of the active compound.

For buccal administration, the compositions may take the form of tablets, muco-adhesive patches/stickers or lozenges formulated in conventional manner.

The compositions may also be formulated in rectal compositions such as suppositories or retention enemas, e.g., containing conventional suppository bases such as cocoa butter or other glycerides.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin or glycerol, for use in an inhaler or insufflator may be formulated containing a powder mix of the compound and a suitable powder base such as lactose or starch.

The term “treating” as used herein refers to means of obtaining a desired physiological effect. The effect may be therapeutic in terms of partially or completely curing a disease and/or symptoms attributed to the disease. The term refers to inhibiting the disease, i.e. arresting its development; or ameliorating the disease, i.e. causing regression of the disease.

While certain features of the present application have been illustrated and described herein, many modifications, substitutions, changes, and equivalents will be apparent to those of ordinary skill in the art. It is, therefore, to be understood that the appended claims are intended to cover all such modifications and changes as fall within the true spirit of the present application.

Applications of Cell Painting Platform:

The platform enables the end user to modify cellular surface of target cells using liposomes with different functional groups or directly by means of chemical modification.

Target Cell Labelling (In-Vivo Cell Modification):
1.1. Anti-cancer:
   1.1.1. Labelling cancer cells for killing by immune system cells. For example, by presenting an alkyne group on cancer cells and systemic injection of alkyne or azide-anti-CD3 and alkyne or azide-anti-CD8 to induce cancer killing by killer T cells.
   1.1.2. Labelling cancer cells for killing by anti-cancer peptides. For example, by presenting alkyne or azide group, respectively, on cancer cells and by injecting azide-anti-cancer peptide that require membranal anchor for cell killing.
1.2. Anti-autoimmune diseases: labelling self-reactive cells for killing by immune system cells.

2. Effector Cell Labelling (Ex-Vivo Cell Modification):
2.1. Patient derived killer T-cells can be labeled ex-vivo with a new group that allows target cell recognition, followed by target cell killing. For example, anti-CD3-alkyne or azide can be covalently linked to targeting peptide-azide or alkyne (epitope) or anti-CD19 antibody-azide or alkyne, that can be used to treat B cell lymphoma, or HLA-MART1 antibody-azide for killing melanoma cells, or anti GP120 antibody-azide or alkyne for killing HIV infected T cells.
2.2. Primary regulatory T cells can be labeled using anti-CD3-alkyne or azide that can be covalently bound to chronic inflammation site targeting antibody-azide or alkyne or peptide-azide or alkyne to inhibit inflammation progression for MS, arthritis, psoriasis, etc.
2.3. Circulating tumor cell modification: tumor cells can be labeled with immune activating moiety that will cause activation of immune effector cells against these cells resulting in novel cancer vaccine formulation.

The invention will now be illustrated by the following non-limiting examples.

### EXAMPLES

### Materials and Methods:

### Production of Immune Labelling Liposomes Using Ethanol Injection:

Lipids (Avanti-polar lipids or lipoid) were weighed according to the required composition and were solubilized in EtOH absolute at final volume of 10% of the required liposome volume. Lipids-EtOH mixture was heated above the Tm (melting temperature) of the lipids. EtOH injection was performed into the appropriate buffer at identical temperature and lipid-buffer was mixed and extruded to yield liposomes at the desired size distribution using an extruder.

### Liposome Modification Post Production:

Liposomes containing ethanolamine group were chemically modified post extrusion with a linker and azide (one member of a binding pair) using the NHS ester chemical reaction (N-hydroxysuccinimide). Typically, the NETS-polyethylene glycol (PEG)4-Azide (NHS group) is used at 5 molar equivalents per primary amine group (DOPE lipid). The unbound excess was removed using size exclusion chromatography.

Liposomes were alternatively made using a pre-modified lipid to yield a similar liposomal product that allows a copper dependent or independent click reaction. Briefly, a DSPE or DOPE lipid pre-modified with PEG4-alkyne or azide was incorporated into lipid mixture prior to EtOH injection.

PEG4 represents PEG having a molecular weight of about 194 Da.

### Antibody Modification:

Antibodies are routinely modified and cleaned using the same method as for liposome chemical modification with slight modifications. Briefly, a 50-molar excess of NHS-PEG4-BCN (the other member of this binding pair) is added per antibody. Unbound excess was removed using size exclusion chromatography.

### Creating 2STEP, OUT, IN+OUT, IN Approaches Using Modified Antibodies and Modified Liposomes:

2STEP: Liposomes covalently linked to one member of the binding pair (e.g. azide), were used directly on cells at the appropriate dilution (or injected IV under animal models) followed by washes of treated cells (not applicable under in-vivo settings) and were allowed to react with antibodies modified with the complementary member of the binding pair (e.g. BCN). For detection purposes, immune-liposome labeled cells were allowed to react fluorescent dye with the complementary member of the binding pair (e.g. DBCO).

OUT: Liposomes covalently linked to one member of the binding pair (e.g. azide), were allowed to react with antibodies with the complementary member of the binding pair (e.g. BCN). Modified liposomes were then used directly on cells at the appropriate dilution (or injected IV under animal models) followed by washes of treated cells (not applicable under in-vivo settings). For detection purposes, immune-liposome labeled cells were allowed to react fluorescent dye with the complementary member of the binding pair (e.g. DBCO).

IN+OUT: Lipids covalently linked to one member of the binding pair, were mixed with antibodies with the complementary member of the binding pair before extrusion. Liposomes were then created using extruder and allowed reaction to complete (18 hrs at 400 RPM at 25° C.). Modified liposomes were used directly on cells at the appropriate dilution (or injected IV under animal models) followed by washes of treated cells (not applicable under in-vivo settings) and were allowed to react with a fluorescent dye with the complementary member of the binding pair.

IN: Lipids covalently linked to one member of the binding pair, were mixed with antibodies with the complementary member of the binding pair and required catalysts or reagents before extrusion. Liposomes were then created using extruder and cleaned immediately using size exclusion or dialysis to inhibit reaction with antibodies on the outer leaflet. Inner leaflet reaction was allowed complete in a catalyst- or reagent-free buffer (18 hrs at 400RPM at RT). Modified liposomes were used directly on cells at the appropriate dilution (or injected IV under animal models) followed by washes of treated cells (not applicable under in-vivo settings) and were allowed to react with a fluorescent dye with the complementary member of the binding pair.

### Cell Growth and Selection:

Cell lines are grown at 37° C. under 5% CO₂ using the medium recommended by the ATCC, typically RPMI or DMEM, supplemented with penicillin and streptomycin, amphotericin B, heat inactivated bovine calf serum, and L-Glutamine. Cells are harvested using trypsin solution in HBSS, for 5-10 minutes at 37° C., collected using pipette, and centrifuged at 400 g for 5 minutes. Pelleted cells are re-suspended in pyrogen free PBS⁻⁻ buffer (without calcium and magnesium) or in growth medium and are then counted a hemocytometer under a phase contrast microscope, using trypan blue as live-dead discriminating dye. Cells are sub-cultured up to 10 passages and are routinely tested for mycoplasma.

### Flow Cytometry-Based Cell Painting Analysis of Single Cells:

Typically, 500,000 cells are used per tube, and experiments are done with two biological repeats, in triplicates. Cells were incubated with FITC-labeled liposomes at 0.5 mM lipids for the required time (typically 1 hr) at 37° C. under 5% CO₂ in growth medium. Cells are then washed 3 times using in pyrogen free PBS⁻⁻. Cells are later stained using DBCO-Cy5 (a clickable fluorescent dye) for 1 hr in PBS⁻⁻. Cells undergo 3 more washes with PBS⁻⁻ and are fixed using PFA at 1.6% in PBS⁻⁻ for 15 minutes and washed and re-suspended in PBS⁻⁻. Fixed cells are stored at 4° C. for several minutes up to 7 days prior to FACS analysis using BD FACSCalibur.

Cells are analyzed using manual gating of the side scatter and forward scatter detected signals and are gated accordingly, to distinguish between intact cells and debris. 10,000 cells are counted per tube and analyzed using the required fluorescent channels: FL1 channel: green fluorescent channel (530±15 nm, FL1). Laser used is 488 nm, 15 mW; FL4 channel: red fluorescent channel (661±8 nm, FL4). Laser used is 635 nm, 9 mW. Signal threshold is determined using control liposomes (or no liposome) treated cells, set gate above fluorescence signal of unstained control to determine positive signal, and calculate percent of positive cells.

### Isolation of Primary Killer T Cells:

Primary mouse splenocytes or venous blood supplemented with tri-sodium citrate (diluted 1:9 citrate 0.11M to blood) were separated using pyrogen free Ficoll (1.077) and were primed using IL2 and anti-CD3 and anti-CD28 for 5-13 days at 37° C. under 5% CO₂. They were used as a source of primary effector/memory killer T cells²⁰.

### CFSE Staining of Cells:

For flow cytometry use: 1 μl of CFSE stock (2.5 mg/ml in DMSO) is added to 1 ml of growth medium containing 1 to 8 million cells. Cells are immediately vortexed and incubated for 30 minutes in tissue culture incubator. Stained cells are washed 3 times using growth medium (1 wash: cells are centrifuged at 400 g for 5 minutes, pelleted cells are re-suspended in medium).

Modified protocol for fluorescent microscopy: 1 μl of CFSE stock (2.5 mg/ml in DMSO) is added to 1 ml of pyrogen free PBS containing 1 to 8 million cells. Cells are immediately vortexed and incubated for 30 minutes in tissue culture incubator. Stained cells are washed 3 times using growth medium.

### Imaging Immune-Liposomes Labeled Cancer Cell Killing by Primary Immune Cells:

4T1mCherry cells were treated with N8 liposomes (modified with PEG4-azide) at 5 mM lipids for 1 hr at 37° C. under 5% CO₂. Cells were washed 3 times using pyrogen free PBS and were allowed to react with antibodies labeled using PEG4-BCN in growth medium for 1 hr at a ratio of 12.5 μg each mAb per 100 μl of 100 mM lipids. The cancer cells were co-incubated CFSE stained primary killer T cells at 37° C. under 5% CO₂ and were imaged every 5 minutes at the green and the red channels of the LSM 710 confocal microscope, for the duration of 24 hrs. Control was done without exposing the cancer cells to the liposomes, but using the same donor killer T cells, under identical conditions.

### In-Vivo Orthotropic Triple Negative Breast Cancer Model Induction in Mice:

Mice are obtained from Harlan (Envigo, Israel) and are kept at an SPF (specific pathogen free) facility with 12 hrs light/dark cycles, with food and water ad libitum. All performed experiments were approved by the institutional animal studies ethics committee. 4T1 murine cancer cell line (300,000 cells in 50 μl of PBS⁻⁻) was injected using a 30 G needle to the mammary fat pad of 7-8 week old female balb/C mice. Palpable tumors appear 5-10 days post injection of cells. Typically, treatment commences at average tumor size of 100 mm³. Animals are euthanized at tumor size of 1000 mm³ or if animal losses 15% of the initial body weight, as dictated by the institutional animal studies ethics committee, using CO₂. Tumor size is determined using caliper to measure the longest dimension (L) of the tumor and the dimension perpendicular to it (W). Tumor volume (V) is estimated using the formula below:

V=W*W*L/2

All treatments are given systemically using IV injections to the tumor bearing mice.

Chemical compound structures synthesized and used to obtain the results presented here, and their synthesis protocols are below:

| Compound | structure |
|---|---|
| DOPE/DSPE- PEG200 propargyl | 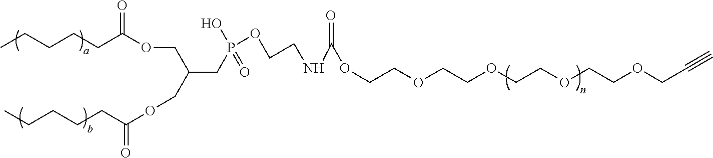 |
| DSPE-PEG200, 400 propargyl | 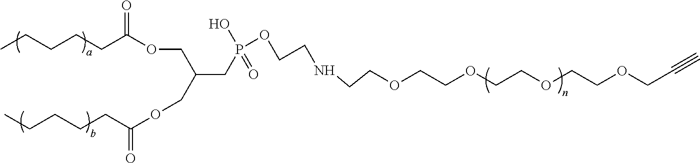 |
| DMPE, DPPE, DSPE-heptynoic acid | 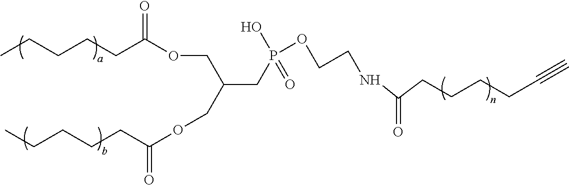 |
| OTS-PEGX- PROPARGYL 200, 400, 600 | 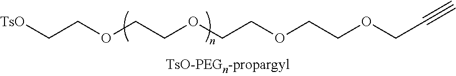 |
| NHS-PEGX- PROPARGYL 200, 400, 600 | 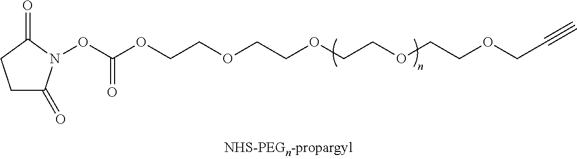 |
| NHS-HEPTYNOIC ACID | 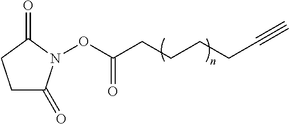 |
| NHS-PEG_{200/1100}-BCN | 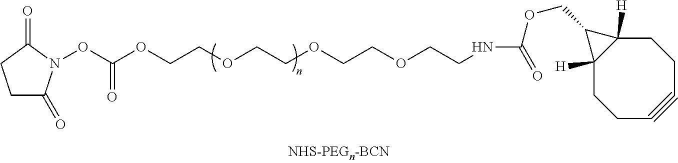 |
| DOPE-PEG₄-200, 600, 1000, 2000-N₃, DSPE-PEG₄-200, 2000N₃, | 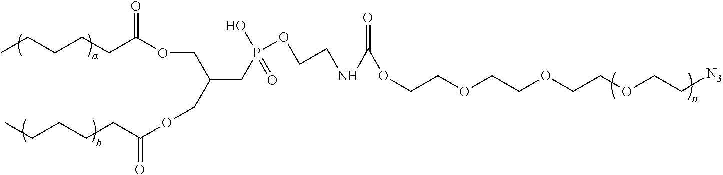 |
| NHS-PEG_{600/1000/2000/200}-N3 | 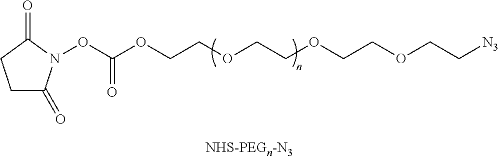 |
| DSPE-PEG200-AZIDE | 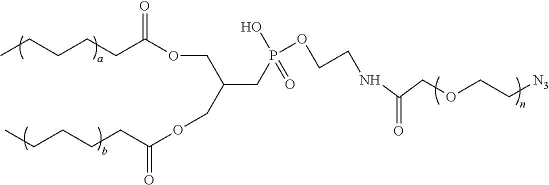 |
| OTS-PEGX-N3 | 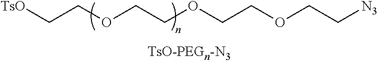 |
| NHS-PEG₂₀₀-BIOTIN | BIOTIN LABELLING LIPIDS AND REAGENTS |
| NHS-NC8-BIOTIN | |
| NHS-BIOTIN | |
| DOPE-FITC | FLUORESCENTLY LABELLED LIPIDS |
| DSPE-FITC | |

### Synthesis of Biotin-NHS:

To 0.72 gr of Biotin (2.94 mmol) 40 ml of dimethylformamide were added followed by addition of 1.69 gr NHS (14.68 mmol). To reaction solution 2.00 gr of DCC (14.80 mmol) were added and reaction was stirred for 18 hours at ambient temperature before completion. Reaction was tested by TLC (TLC mobile phase: 80% Ethyl Acetate: 20% Methanol; Staining PMA). Reaction solution was filtered and then diluted with 100 ml of solution (30% Ethyl Acetate: 70% hexane). The product was filtered to get 600 mg of product contain some traces of reagents. Then additional 50 ml of solution was added to get more precipitation. The precipitate was removed by filtration to get 66 mg of pure product (tested by TLC and NMR). Then to reaction solution additional 100 ml same solution was added to get more precipitate. After isolation 600 mg of product in mixture with some urea byproduct were isolated. Pure product (66 mg) was used for reactions for biological applications.

### Synthesis of BCN-PEG1100-NHS:

To 30 mg of BCN-NHS (0.10 mmol) 5 ml of chloroform were added followed by addition of 0.2 ml triethylamine. To reaction 100 mg of NH2-Peg1100COOH (0.09 mmol) were added and reaction was stirred for 2 hours before it was tested by TLC (TLC mobile phase: 80% Chloroform, 20% Methanol and 100% chloroform; Staining PMA). Reaction was not complete (NH2-PEG1100COOH left). Therefore additional 15 mg of BCN-NHS (0.05 mmol) were added and reaction was stirred for additional 1 hour. Reaction was complete according to TLC test (no NH2-PEG1100COOH left after reaction and new less polar spot is observed). Triethylamine was evaporated by rotovapor. Then reaction was diluted in 3 ml chloroform. The product was precipitated by addition 30 ml of diethyl ether. The product crude was evaporated to give 120 mg and was used in the next step as is.

To 120 mg of BCN-PEG1100COOH (0.09 mmol) 5 ml of acetonitrile were added followed by addition of 0.2 ml triethylamine. To reaction solution 50 mg of DSC (0.20 mmol) were added and reaction was stirred for 2 hours. Reaction was tested by TLC (TLC mobile phase: 80% Chloroform, 20% Methanol; Staining PMA). Reaction was complete (no BCN-PEG1100COOH left after reaction and new less polar spot is observed). Reaction solution was evaporated to dryness by rotovapor under reduced pressure. Then reaction residue was dissolved in 10 ml of solution 5 ml: 5 ml dichloromethane:diethylether. The reaction mixture was stirred for 15 minutes and residue stayed in the flask while reaction solution was evaporated to dryness by rotovapor. The obtained solid—120 mg (yield about 93%) was tested by TLC and NMR. The product was kept in freezer.

### Synthesis of Fluorescent Lipids DSPE-FITC, DOPE-FITC:

To 105 mg of lipid (DSPE or DOPE) 10 ml of chloroform were added followed by addition of 50 mg of FITC and 1.0 ml of triethylamine. Reactions were stirred for 15 minutes at ambient temperature before addition of 5 ml of DMF. Reactions were stirred for 1 hour at ambient temperature and were complete according to TLC (TLC mobile phase: 25% methanol, 75% Chloroform; Staining PMA). Then reactions were diluted*10 by chloroform and purified by flash chromatography. The products were eluted by 30% methanol: 70% chloroform. The pure products fractions were combined and evaporated to dryness. 95 mg of DOPE-FITC and 70 mg of DSPE-FITC were isolated. The product structures were confirmed by NMR, TLC.

Synthesis of alkyne-PEG-DSPE Conjugates.

To 10 gr PEG200 or 20 gr PEG400 (0.05 mole) 150 ml of dry THF were added under inert conditions and solutions were cooled to 0° C. by ice bath. After 15 minutes Sodium hydride 1.8 gr was added in three portions each about 0.6 gr to each reaction. Reactions were stirred for additional 30 minutes at ambient temperature before addition of propargyl bromide (2.8 ml to each reaction). Then reactions were allowed to be stirred overnight. In all cases products were obtained by TLC (5% MeOH: 95% Ethyl Acetate). The reactions were neutralized by addition of 4 ml of HCl 32%, following evaporation to dryness. Then traces of propargyl bromide were removed by hexane wash. Purification of products was done on silica gel flash columns. The products were eluted by Ethyl Acetate to 90% Ethyl Acetate: 10% MeOH. The combined fractions were evaporated to dryness and taken to MS analysis. The best products fractions were used as is in the next steps. In case of PEG200-propargyl fraction was 1.2 gr and in case of PEG400-propargyl fraction was 2.7 gr.

0.5 gr of PEG200-propargyl and 1.0 gr of PEG400-propargyl were added with 10 ml of THF and 2 ml of triethylamine each reaction. Both reactions were stirred for 15 minutes before 0.4 gr of tosyl chloride was added to each reaction. Reactions were complete after overnight stirring according to TLC (20% Methanol: 80% chloroform). To both reactions 2 ml of triethylamine, 0.5 gr of DSPE and 5 ml of chloroform were added and reaction were stirred at 40° C. for overnight till completions as was observed from TLC. Both reactions were filtered to remove insoluble particles and evaporated to dryness. Purification of products was done on silica gel flash columns. Reaction mixtures were dissolved in 5 ml chloroform each and loaded on columns and eluted by gradient till 20% MeOH: 80% chloroform. DSPE-PEG200-propargyl and DSPE-PEG400-propargyl were evaporated to give about 400 mg of each product that were identified by NMR.

### Synthesis of DOPE-PEG2000azide and DSPE-PEG2000azide

To 200 mg of lipid (DSPE or DOPE) 10 ml of chloroform were added followed by addition of 1.50 gr of crude NHS-PEG2000-Azide and 2.0 ml of triethylamine. Reactions were stirred for overnight, reactions were complete according to TLC (TLC mobile phase: 10% methanol, 90% Chloroform; Staining PMA). 400 mg of 2-azidoethyl amine were added and both reactions were stirred for additional 4 hours. Then reactions were evaporated by rotovapor to remove triethylamine and traces of 2-azidoethyl amine. Reactions crudes were diluted with 20 ml of solution 5% MeOH: 95% Chloroform and purified on silica columns. The products were eluted by 8% methanol: 92% dichloromethane. The product fractions with purity ≥90% according to TLC were combined and evaporated to dryness. In order to get pure compound crystallizations were performed. Both products were dissolved in minimal volume of dichloromethane following addition of diethyl ether causes precipitation of some impurities while the products are soluble. Therefore the product solutions were filtered and added with additional diethyl ether till precipitation of the product lipids was observed. Total 280 mg of pure DOPE-PEG2000-Azide and 75 mg of pure DSPE-PEG2000-Azide were obtained after crystallizations. The products structure were confirmed by NMR.

Formation of Conjugate of 6-Heptynoic acid NHS with 14 DMPE, 16 DPPE or 18 DSPE:

To 200 mg of lipid (DPPE, DMPE or DSPE) 10 ml of chloroform were added followed by addition of 2.5 ml of triethylamine. Each reaction was stirred for 15 minutes at ambient temperature before addition of 100 mg of 6-Heptynoic Acid NHS ester. Reactions were stirred for 2 hours at RT and concentrated to minimal volume by rotovapor. Then reactions residues were dissolved in 150 ml of ethyl acetate, diethylether solution (100 ml ethyl acetate, 50 ml diethylether). TLC was done to test conversion level of the reactions (TLC mobile phase: 20% methanol, 80% Chloroform; Staining PMA). In all cases reaction were complete (no lipid left after reaction). Reaction solutions were stirred for 15 minutes with 50 ml of saturated sodium bicarbonate solution, then water layers were removed and organic layers were washed with 50 ml sodium chloride solution. After discarding water layers organic layers were dried with Sodium Sulfate and evaporated to dryness by rotovapor. 226 mg of DPPE-6 heptynoic (yield 98%), 200 mg of DPPE-6 heptynoic (yield 98%) and 150 mg of DSPE-6 heptynoic (yield 70%) of solid products were obtained. All products were identified by NMR and TLC.

### Synthesis of BCN-NHS:

The procedure was done according to published procedure molecules 2013, 18, 7346-7363 with several changes. To 400 mg of BCN-OH (2.66 mmol) 10 ml of acetonitrile were added followed by addition of 1.5 ml triethylamine. To reaction solution 1.70 gr of DSC (6.64 mmol) were added and reaction was stirred under inert conditions. Reaction solution was stirred at ambient temperature for overnight before completion as was observed by TLC (TLC mobile phase: 50% Ethyl Acetate, 50% hexane; Staining PMA). Reaction solution was evaporated to dryness by rotovapor. Then reaction residue was dissolved in 5 ml of chloroform and added with 50 ml of diethyl ether. The reaction mixture was stirred for 15 minutes and residue stayed in the flask while reaction solution was evaporated to dryness by rotovapor. The obtained solid—950 mg (yield about 95%) According to TLC the purity was more than 90-95% therefore the product was used as is the next step/steps.

### Synthesis of BCN-PEG₄-OH:

To 300 mg of BCN-NHS (1.03 mmol) 10 ml of acetonitrile were added followed by addition of 1.0 ml triethylamine. To reaction solution first 0.3 ml of OH-PEG₄-NH₂ was added and reaction was stirred for half hour before it was tested by TLC reaction was not complete. Then additional 0.2 ml of OH-PEG₄-NH₂ were added to total 0.5 ml of OH-PEG₄-NH₂ (2.83 mmol) and reaction was stirred for half hour till completion as was observed by TLC (TLC mobile phase: 90% Chloroform, 10% Methanol; Staining PMA). Reaction solution was evaporated under reduced pressure to dryness by rotovapor followed by purification by silica column. The reaction solution was eluted with 5% MeOH: 95% dichloromethane. The pure fractions were evaporated to dryness by rotovapor. The obtained product—200 mg (53%) was tested by TLC. According to TLC the purity was about 90% therefore the product was used as is the next step/steps.

### Synthesis of BCN-PEG₄-NHS

To 200 mg of BCN-PEG₄-OH (0.54 mmol) 5 ml of acetonitrile were added followed by addition of 1 ml triethylamine. To reaction solution 400 mg of DSC (1.56 mmol) were added and reaction was stirred for 2 hours at ambient temperature till completion as was observed by TLC (TLC mobile phase: 10% Methanol, 90% Dicholoromethane; Staining PMA). Then reaction solution was evaporated to dryness by rotovapor following purification on silica column. The column was washed with dichloromethane and the product was eluted with 100% Ethyl Acetate. The pure fractions were evaporated to dryness by rotovapor. The obtained product—250 mg (91%) was tested by TLC and identified by TLC and NMR. According to TLC and NMR the purity was more than 95% therefore the product was used as is the next step. The product was kept at −20° C.

Formation of Tetraethylene Glycol p-toluenesulfonate (HOPeg₄OTs)

To 55 gr of peg₄ (0.283 mole) 400 ml of dry chloroform were added and solutions were cooled to 0° C. by ice bath. After 15 minutes 100 ml of triethylamine were added and reaction was stirred additional 15 minutes. Then 25 gr of tosyl chloride (0.132 mole) were added and reaction was stirred for overnight at ambient temperature. Conversion was tested by TLC after overnight (100% Ethyl Acetate). Then reaction was evaporated by rotovapor following wash of residue with hexane:ether (2:1) solution to remove traces of tosyl. Then residue was added with ethyl acetate and washed with water solutions of 5% HCl, 10% NH₄Ac and 5% NaCl followed by drying of organic layer with Sodium Sulfate. Ethyl acetate was evaporated by rotovapor to give 18.6 gr of HOpeg₄OTs (yield 41%). According to TLC the purity was about 90% therefore the product was used as is the next step/steps. The product was kept at 4° C.

Formation of Tetraethylene Glycol Azide (HOPEG₄N₃)

To 6.0 gr of HOPEG₄OTs (17.2 mmol) 60 ml of ethanol were added and solution was stirred for 5 minutes. Then 6.0 gr of sodium azide (92.3 mmol) were added and reaction mixture was heated to 65° C. and stirred for overnight till completion as was observed by TLC (TLC mobile phase: 90% Chloroform, 10% Methanol; Staining PMA). Then the mixture was filtered to remove insoluble sodium azide. The ethanol solution was evaporated by rotovapor and the residue was dissolved in diethyl ether. The product containing ether layer was filtered and concentrated by rotovapor to give crude product following additional purification by flash silica column. The product was eluted with 5% MeOH: 95% Chloroform. Pure fractions (≥90% purity by TLC) were combined and evaporated to dryness to give 0.50 gr of HOPeg₄N₃ that was used as is the next step/steps.

Formation of N₃-peg₄-NHS:

To 450 mg of HOPeg₄N₃ (2.05 mmol) 15 ml of acetonitrile were added followed by addition of 1 ml triethylamine. To reaction solution 800 mg of DSC (3.12 mmol) were added and reaction was stirred for overnight at ambient temperature till completion as was observed by TLC (TLC mobile phase: 10% Methanol, 90% Dicholoromethane; Staining PMA). Reaction solution was evaporated to dryness by rotovapor following dissolving the residue in dichloromethane. The combined dichloromethane layers were concentrated to 3 ml and added with 30 ml of petroleum ether. The reaction mixture was stirred for 15 minutes and petroleum ether solution was disposed. The residue was dissolved in dichloromethane: diethyl ether (10 ml each, 1:1). The obtained solution was evaporated to dryness to give 370 mg of NHSPeg₄N₃ (yield 50%). According to TLC and NMR the purity was more than 95% therefore the product was used as is the next step. The product was kept in freezer at −20° C.

### Example 1. Labelling of Cells Using Fusogenic Liposomes

In vitro:

An immune labelling liposomal platform was developed, using different lipids with various Tm (melting temperatures) values as determined by the saturation (presence of double bonds) and length of the acyl tail. Combination of such lipid compositions with different proportions of positively charged and zwitterionic lipids (such as DAPE, diacyl phosphatidylethanolamine) significantly improved the fusion with cancer cells. Our liposomal labelling platform was used to label cancer cells with one functional group of a binding pair, such as click chemistry (FIG. 2A). This functional group is used to add an immune activating agent, such as monoclonal antibody (mAb) using several chemical synthesis steps (examples presented in FIGS. 2B and C) to allow addition of clickable linkers to both phospholipid head group and to mAb.

### Example 2. Liposomal Composition, Liposomal Uptake and Liposomal Fusion

Liposomes prepared with the following formulation HSPC:cholesterol:6-Heptynoic-PE (DSPE/DPPE/DMPE) 60:35:5 were produced and found to be stable.

All materials not synthesized as described, are commercially available. Liposomes prepared with the following formulation: HSPC:cholesterol:6-Heptynoic-PE (DSPE/DPPE/DMPE) 60:35:5 were produced and found to be stable.

HSPC—hydrogenated soy phosphatidylcholine;

PE—phosphatidylethanolamine

DSPE—distearoylphosphatidylethanolamine

DPPE—1,2-dipalmitoyl-sn-glycero-3-phosphoethanolamine

DMPE—1,2-Dimyristoyl-sn-glycero-3-phosphoethanolamine

The 6-Heptynoic acid linker is a sample of a copper dependent alkyne used for preparation of liposomes with mAbs bound only to inner leaflet. This linker was later replaced with copper free alkyne linkers. BCN or DBCO are copper independent alkyne groups that enable covalent bond formation with azide under in-vivo conditions.

As a first step 6-Heptynoic acid was conjugated to NHS to create a bi-functional linker with NHS and alkyne groups. Using this linker, the amine group on PE was conjugated to 6-Heptynoic-NHS through the NHS group and the functionalised PE was purified for liposome preparations.

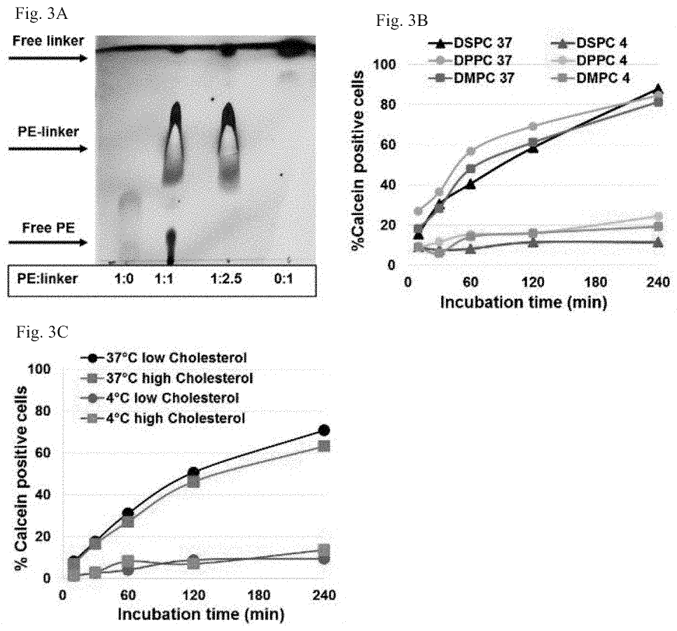

These liposomes bearing an alkyne functionalised linker can be used for conjugation of various azide modified molecules such as peptides, antibodies, fluorophores, biotin, and saccharides. We conjugated a fluorophore, calcein-azide (in-house production) to the liposomes containing the alkyne linker (FIG. 3 A). The conjugation efficiency was 17-20%. These fluorescent liposomes were used to test the effect of different liposome formulations on cellular uptake or fusion with liposomes. The length of the hydrophobic tail and the percentage of cholesterol in the liposome formulation did not have a significant effect on liposome uptake or fusion by cells (FIG. 3B-C).

Head group modification affects the liposomes' effect on target cells: by modifying the head group we can fine tune the efficacy of liposome-target cell fusion versus liposomal uptake by endocytosis.

4T1 cell line (mouse triple negative breast cancer cells available from the ATCC (ATCC® CRL-2539™) were investigated for the target cell labelling using our platform. 4T1 cells were incubated with novel formulations of fluorescently labeled liposomes that enhance fusion with target cells. The fusion with target cells is determined by using different linkers that are bound to outer liposome membrane leaflet or to both inner and outer leaflets of the liposome.

### Example 3. Liposomal Composition Effect on Cancer Cell Fusion

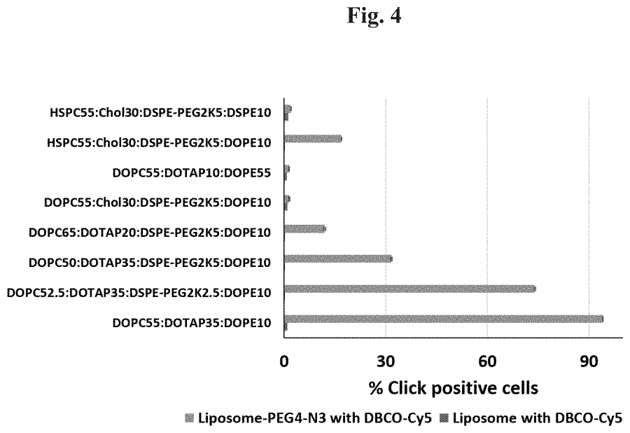

Different lipid compositions were tested as liposomes for their ability to fuse with cancer cells as illustrated in FIG. 2D but using a liposome without DOPE-FITC. We modified the net positive charge, acyl tail saturation and therefore the Tm (melting temperature) of the lipid mixture. We have obtained tunable cancer labelling liposomes, that added a functional group (one member of the binding pair) on the cancer cells' membrane (FIG. 4).

### Example 4. Formulation Optimization

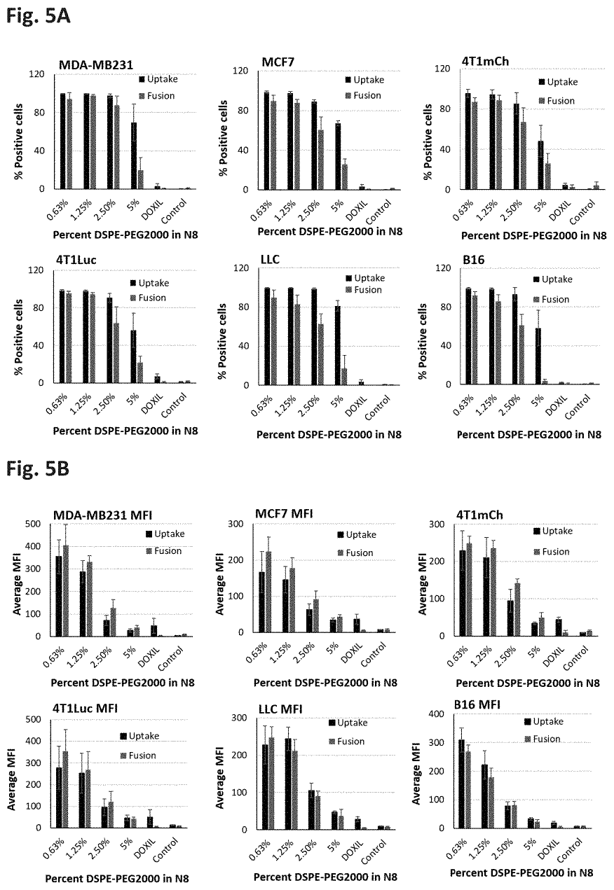

DSPE-PEG2000 is used as a stabilizer and improves circulation half life time under in-vivo conditions but could also result in reduction of cancer-liposome fusion due to steric hindrance. Therefore, we have tested a broader range of DSPE-PEG2000 in our liposomal immune labelling formulation N8, core formulation DOTAP:DOPC: DOPE:DOPE-FITC:DSPE-PEG2K 35:52.5:10:0.2:X where X is 5, 2.5, 1.25, 0.625, molar ratio). In order to determine the effect of PEG2000 on uptake and fusion with cancer cells, the liposomes were fluorescently labeled using DOPE-FITC (ex 488 nm, em 530 nm) and were connected to the linker PEG4-N₃ post production using NETS-PEG₄-N₃ (FIG. 2D). Cancer cells were exposed to liposomes at 0.5 mM lipids for 1 hr at 37° C., washed and stained using DBCO-Cy5 (FL4). Signal in green fluorescent channel (FL1) is indicative of liposomal uptake by cancer cells. Signal in red fluorescent channel (FL4) is indicative of fusion of our painting liposomes with cancer cell's membrane. Cells were fixed using paraformaldehyde 1.6% in PBS for 15 minutes at room temperature and were kept at 4° C. until FACS analysis. FIG. 5A presents the averaged percent of positive cancer cells to the FITC signal, indicating uptake of liposomes, and to the Cy5 signal, clicked onto the azide group on those cells, thus indicating fusion. Furthermore, in FIG. 5B, the averages of mean fluorescent intensity are presented and is proportional to the number of fluorophores per cancer cell. Collectively, elevated quantities of DSPE-PEG2000 in the immune labelling formulation presented an inhibition in both fusion and uptake.

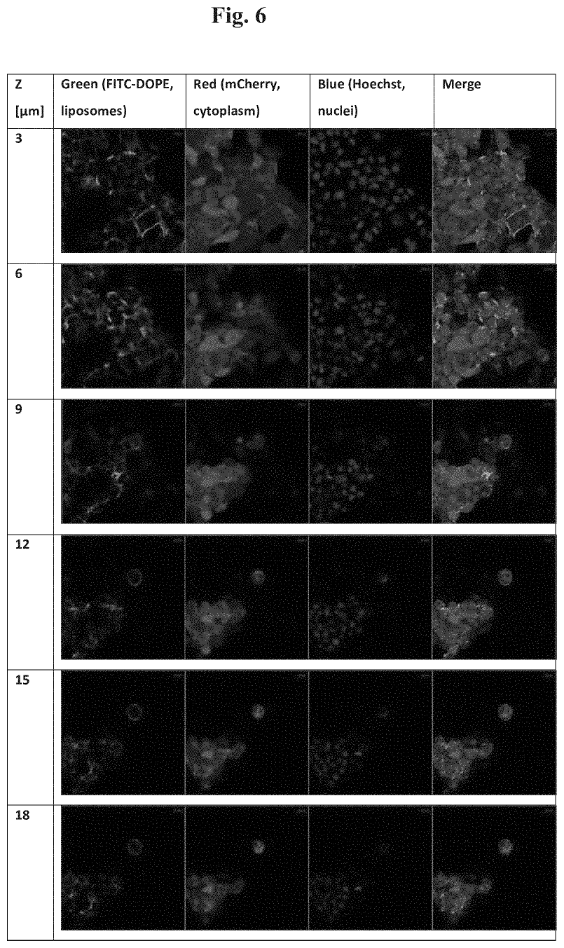

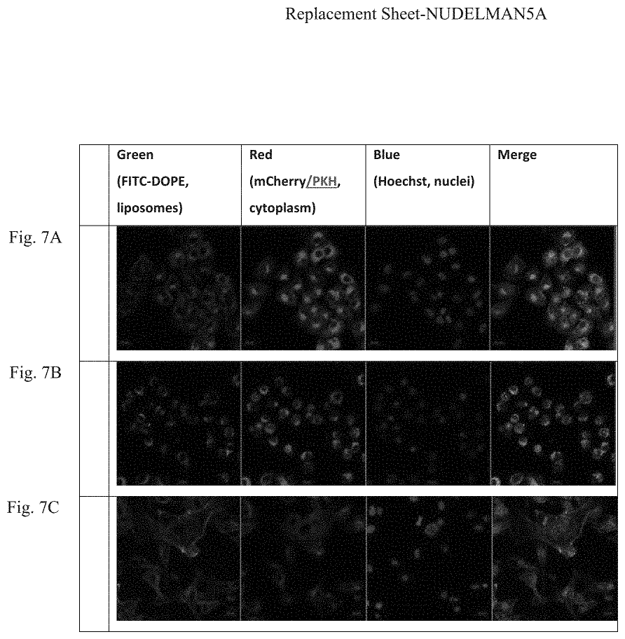

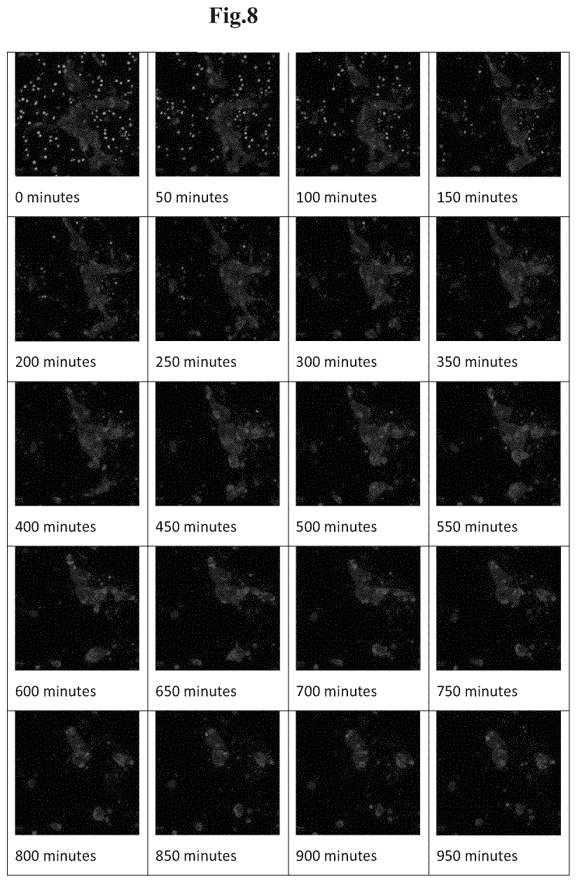

In order to complement the abovementioned results obtained using flow cytometry, we have tested the localization of our fluorescently labeled liposomes in different cancer cells. FIG. 6 presents the spatial localization of the immune-labelling liposomes to the membrane of 4T1mCherry cells which correlates well with membranal localization of our lipids. We have tested our ability to immune-label other cancer cell types, as can be seen in FIG. 7 for lung cancer cell lines (human and murine) a melanoma cell line (murine).

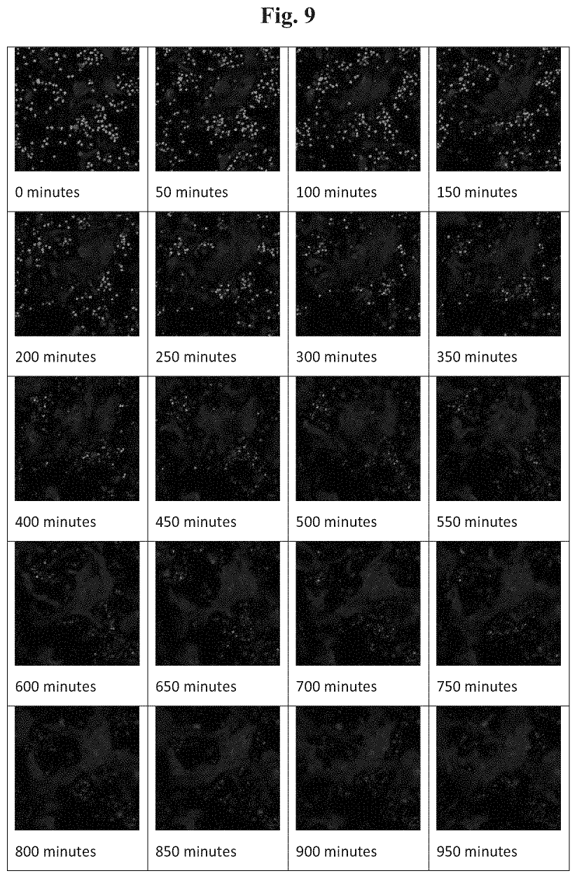

Next, we tested the ability of our immune labelling liposomes to induce cancer cell death by activation of killer T cells. This was performed using confocal time lapse experiments for treated cancer cells versus untreated cancer cells with mouse primary killer T cells from same donor presented in (FIG. 8 and FIG. 9 respectively). We have performed image quantification using the red pixels as a marker for cancer cell progression/killing (10).

We have used four different approaches for the delivery of killer T cell activating mAbs to the tumor. The first approach we used is named “2STEP”, where liposome described in (FIG. 2A) is injected and labels the cancer cells with one functional group of the binding pair (for example azide group) and 3 hrs post liposome injection, we injected the mAb labeled with the other functional group of the binding pair. The second approach (FIG. 2E, I) is named “IN” where the inner leaflet is used for binding the mAb or mAbs covalently bound to the other binding pair functional group. The third approach, “OUT” (FIG. 2E, II), uses the outer leaflet of the liposome to bind the mAb or mAbs covalently bound to the other binding pair functional group. The fourth approach, “IN+OUT” (FIG. 2E, III) uses both inner and outer leaflets of the liposome to bind the mAb or mAbs covalently bound to the other binding pair functional group.

### Example 5. Treatment of Cancer in Animal Models

We have tested the efficacy of our immune-labelling liposomes using 2STEP, IN, and IN+OUT approaches versus 2STEP control, (same 2STEP liposomes, coupled with unclickable antibodies) as presented in FIG. 11A. Individual mice spider plots are presented in FIG. 11B, where tumor size versus time per mouse is presented as a single series in each treatment group graph. We have further tested the biological distribution of our liposomal formulations in tumor bearing animals at 24 hrs post injection (FIG. 11C). The liposomal labelling bio-distribution profile is similar to that of the DOXIL formulation used as a benchmark or “Gold standard”.

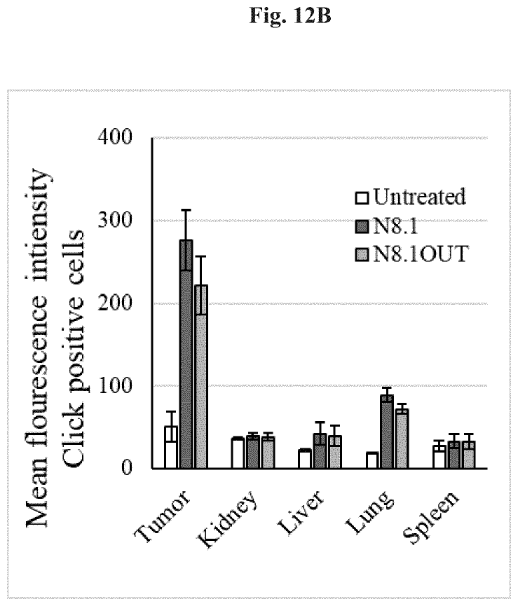

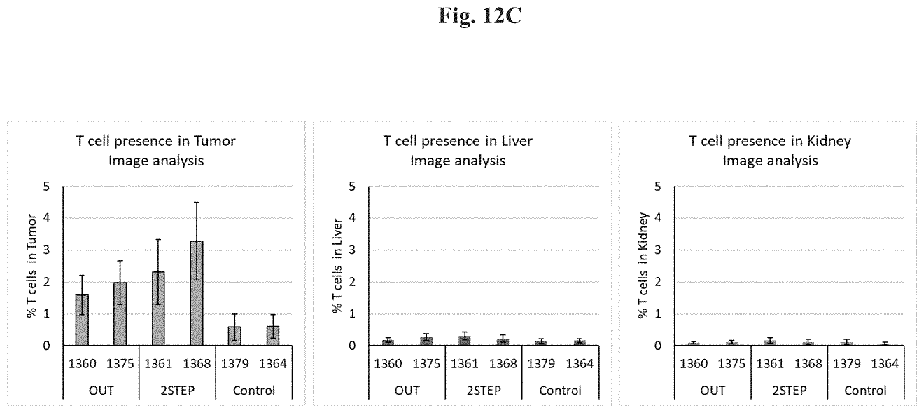

Histological and immuno-histochemical analyses were performed on mice at 72 hrs from treatment in order to test for tumor and other tissue damage as well as T cell recruitment. Data show significant T cell recruitment to tumors but not to liver and kidneys (damage analysis is pending caspase and TUNEL staining). The in-vivo studies were complemented using an ex-vivo selectivity study where organs from 4T1 tumor bearing mice were harvested and digested into single cells. These normal tissue and tumor originating cells were exposed to N8 (2STEP or OUT) liposomes at 0.5 mM lipids and were stained using a clickable dye (DBCO-Cy5). The single cells were analyzed using flow cytometry presented in FIG. 12B. This data when considered alongside the bio-distribution profiles of N8 (2STEP or OUT) and the histological analyses show that our liposomal platform reaches several organs, but fuses selectively and recruit T cells to tumors.

Tumor bearing animals treated with our liposomes have shown increased T cell recruitment to tumors but not to livers or kidneys as seen in FIG. 12A and quantified in FIG. 12C for 2STEP and OUT approaches. The data shown here when combined with the ex-vivo selectivity study, show that the liposomes are selective towards fusion with tumor derived cells and show little fusion with healthy organ derived primary cells (FIG. 12B). The data when combined with the bio-distribution study explains why the liver tissue slides present no increase in T cell infiltration. Mechanistically, the liposomes fuse with negatively charged cancer cells, and activate killer T cells, that recruit additional T cells to the cancer site via chemotaxis. Upon uptake by phagocytes, such as Kuepfer cells in the liver, there is no fusion but there is liposome presence, which on its own, does not activate the killer T cells as for the cancer tissue.

Collectively, our data show that the invention described herein can be used for different cancer types systemically and induces immune reaction aimed at killing the tumor cells under in-vivo conditions.

### REFERENCES

1 Rosenberg, S. A., Spiess, P. & Lafreniere, R. A new approach to the adoptive immunotherapy of cancer with tumor-infiltrating lymphocytes. Science 233, 1318-1321 (1986).
2 de Visser, K. E., Eichten, A. & Coussens, L. M. Paradoxical roles of the immune system during cancer development. Nat Rev Cancer 6, 24-37, doi:10.1038/nrc1782 (2006).
3 Patel, S. P. & Kurzrock, R. PD-L1 Expression as a Predictive Biomarker in Cancer Immunotherapy. Mol Cancer Ther 14, 847-856, doi:10.1158/1535-7163.MCT-14-0983 (2015).
4 Rheinbay, E. et al. Recurrent and functional regulatory mutations in breast cancer. Nature 547, 55-60, doi:10.1038/nature22992 (2017).
5 Schumacher, T. N. & Schreiber, R. D. Neoantigens in cancer immunotherapy. Science 348, 69-74, doi:10.1126/science.aaa4971 (2015).
6 Ali, H. R., Chlon, L., Pharoah, P. D., Markowetz, F. & Caldas, C. Patterns of Immune Infiltration in Breast Cancer and Their Clinical Implications: A Gene-Expression-Based Retrospective Study. PLoS Med 13, e1002194, doi:10.1371/journal.pmed.1002194 (2016).
7 Sharma, P., Hu-Lieskovan, S., Wargo, J. A. & Ribas, A. Primary, Adaptive, and Acquired Resistance to Cancer Immunotherapy. Cell 168, 707-723, doi:10.1016/j.ce11.2017.01.017 (2017).
8 Lee, D. W. et al. T cells expressing CD19 chimeric antigen receptors for acute lymphoblastic leukaemia in children and young adults: a phase 1 dose-escalation trial. Lancet 385, 517-528, doi:10.1016/S0140-6736(14)61403-3 (2015).
9 Dudley, M. E. et al. Cancer regression and autoimmunity in patients after clonal repopulation with antitumor lymphocytes. Science 298, 850-854, doi:10.1126/science.1076514 (2002).
10 Topalian, S. L. et al. Safety, activity, and immune correlates of anti-PD-1 antibody in cancer. N Engl J Med 366, 2443-2454, doi:10.1056/NEJMoa1200690 (2012).
11 Herbst, R. S. et al. Pembrolizumab versus docetaxel for previously treated, PD-L1-positive, advanced non-small-cell lung cancer (KEYNOTE-010): a randomised controlled trial. Lancet 387, 1540-1550, doi:10.1016/S0140-6736(15)01281-7 (2016).
12 Barenholz, Y. Doxil(R)—the first FDA-approved nano-drug: lessons learned. J Control Release 160, 117-134, doi:10.1016/j.jconrel.2012.03.020 (2012).
13 Utsugi, T., Schroit, A. J., Connor, J., Bucana, C. D. & Fidler, I. J. Elevated expression of phosphatidylserine in the outer membrane leaflet of human tumor cells and recognition by activated human blood monocytes. Cancer Res 51, 3062-3066 (1991).
14 Alves, A. C., Ribeiro, D., Nunes, C. & Reis, S. Biophysics in cancer: The relevance of drug-membrane interaction studies. Biochim Biophys Acta 1858, 2231-2244, doi:10.1016/j.bbamem.2016.06.025 (2016).
15 Held-Kuznetsov, V., Rotem, S., Assaraf, Y. G. & Mor, A. Host-defense peptide mimicry for novel antitumor agents. FASEB J 23, 4299-4307, doi:10.1096409-136358 (2009).
16 Colombo, M. P. & Piconese, S. Regulatory-T-cell inhibition versus depletion: the right choice in cancer immunotherapy. Nature reviews. Cancer 7, 880-887, doi:10.1038/nrc2250 (2007).
17 Pardoll, D. M. The blockade of immune checkpoints in cancer immunotherapy. Nat Rev Cancer 12, 252-264, doi:10.1038/nrc3239 (2012).
18 Ohaegbulam, K. C., Assal, A., Lazar-Molnar, E., Yao, Y. & Zang, X. Human cancer immunotherapy with antibodies to the PD-1 and PD-L1 pathway. Trends in molecular medicine 21, 24-33, doi:10.1016/j.molmed.2014.10.009 (2015).
19 Batzri, S. & Korn, E. D. Single bilayer liposomes prepared without sonication. Biochimica et Biophysica Acta (BBA)-Biomembranes 298, 1015-1019 (1973).
20 Oren, R. et al. Functional comparison of engineered T cells carrying a native TCR versus TCR-like antibody-based chimeric antigen receptors indicates affinity/avidity thresholds. The Journal of Immunology 193, 5733-5743 (2014).

1. A method for treating cancer, comprising administering to a cancer patient in need the fusogenic liposome of claim 52.

2. The fusogenic liposome of claim 54, wherein said immune system activating agent is bound via said second functional group to the first functional group of at least one of said lipid molecules at the outer leaflet of the fusogenic liposome.

3. The fusogenic liposome of claim 54, wherein said immune system activating agent is bound via said second functional group to the first functional group of at least one of said lipid molecules at the inner leaflet of the fusogenic liposome.

4. The fusogenic liposome of claim 54, wherein said immune system activating agent is bound via said second functional group to the first functional group of at least one of said lipid molecules at both the outer and inner leaflet of the fusogenic liposome.

5. The fusogenic liposome of claim 54, wherein the immune system activating agent is selected from the group consisting of a T-cell activating agent; a pro-inflammatory cytokine; a memory killer T cell activating peptide; a soluble human leukocyte antigen (sHLA) presenting a viral peptide; and a super-antigen.

6. The fusogenic liposome of claim 5, wherein the immune system activating agent is a T-cell activating agent.

7. The fusogenic liposome of claim 6, wherein the T-cell activating agent is selected from the group consisting of anti-CD3 antibody, an anti-CD8 antibody, an anti-NKG2D antibody, or a combination thereof, an antibody capable of binding both CD3 and CD8 and an antibody capable of binding both CD3 and NKG2D.

8. (canceled)

9. The fusogenic liposome of claim 52, wherein said at least one of said lipid molecules comprising a cationic group is selected from the group consisting of 1,2-dioleoyl-3-trimethylammoniumpropane chloride (DOTAP), dioctadecylamidoglycylspermine (DOGS), 1,2-di-O-octadecenyl-3-trimethylammonium propane (DOTMA), Dimethyldioctadecylammonium (18:0 DDAB), and N1-[2-((1S)-1-[(3-aminopropyl)amino]-4-[di(3-amino-propyl)amino]butyl-carboxamido)ethyl]-3,4-di[oleyloxy]-benzamide (MVL5).

10. The fusogenic liposome of claim 9, wherein said at least one of said lipid molecules comprising a cationic group is DOTAP.

11. The fusogenic liposome of claim 52, wherein said synthetic polymer is selected from the group consisting of polyethyleneimines (PEI) and poly(2-(dimethylamino)ethyl methacrylate.

12. The fusogenic liposome of claim 52, wherein said natural polymer is chitosan.

13. The fusogenic liposome of claim 52, wherein said amino sugar is glucosamine.

14. The fusogenic liposome of claim 52, wherein said cationic polyamino acid is selected from the group consisting of poly(L-lysine), poly(L-arginine), poly(D-lysine), poly(D-arginine), poly(L-ornithine) and poly(D-ornithine).

15. The fusogenic liposome of claim 52, wherein said amphiphilic cancer-cell binding peptide is selected from the group consisting of Cecropin A; Cecropin A 1-8; and cyclic CNGRC.

16. The fusogenic liposome of claim 52, wherein said at least one of said lipid molecules is a phospholipid selected from the group consisting of a phosphatidylcholine, a phosphatidylethanolamine, a phosphatidylserine, a phosphatidic acid and any combination thereof, each one of which comprises one or two identical or different fatty acid residues, wherein the fatty acid residues in the phosphatidyl moiety are saturated, mono-unsaturated or poly-unsaturated and have a carbon chain length of 14, 15, 16, 17, 18, 19, 20, 21, 22, 23 or 24 carbons.

17. The fusogenic liposome of claim 16, wherein said phospholipid is selected from the group consisting of 1-palmitoyl-2-oleoyl-sn-glycero-3-phosphocholine (POPC) and 1,2-dioleoyl-3-phosphatidylethanolamine (DOPE); 1,2-dimyristoyl-3-phosphatidylcholine (DMPC); 1,2-distearoyl-3-phosphatidylcholine (DSPC); 1,2-dimyristoleoyl-sn-glycero-3-phosphocholine (14:1 (Δ9-Cis) PC); 1,2-dimyristelaidoyl-sn-glycero-3-phosphocholine (14:1 (Δ9-Trans) PC); 1,2-dipalmitoleoyl-sn-glycero-3-phosphocholine (16:1 (Δ9-Cis) PC); 1,2-dipalmitelaidoyl-sn-glycero-3-phosphocholine (16:1 (Δ9-Trans) PC); 1,2-dipetroselenoyl-sn-glycero-3-phosphocholine (18:1 (Δ6-Cis) PC); 1,2-dioleoyl-3-phosphatidylcholine (18:1 (49-Cis) PC (DOPC)); 1,2-dielaidoyl-sn-glycero-3-phosphocholine (18:1 (Δ9-Trans) PC); 1,2-dilinoleoyl-sn-glycero-3-phosphocholine (18:2 (Cis) PC (DLPC)); 1,2-dilinolenoyl-sn-glycero-3-phosphocholine (18:3 (Cis) PC); 1,2-dieicosenoyl-sn-glycero-3-phosphocholine (20:1 (Cis) PC); 1,2-diarachidonoyl-sn-glycero-3-phosphocholine (20:4 (Cis) PC); 1,2-didocosahexaenoyl-sn-glycero-3-phosphocholine (22:6 (Cis) PC); 1,2-dierucoyl-sn-glycero-3-phosphocholine (22:1 (Cis) PC); 1,2-dinervonoyl-sn-glycero-3-phosphocholine (24:1 (Cis) PC); 1,2-dimyristoyl-3-3-phosphatidylethanolamine (DMPE); 1,2-dipalmitoyl-3-phosphatidylethanolamine (DPPE); dipalmitoylphosphatidylcholine (DPPC); 1,2-dioleoyl-3-phosphatidylethanolamine (DOPE); 1,2-distearoyl-3-phosphatidylethanolamine (DSPE); 1,2-dimyristoyl-3-phosphatidylserine (DMPS); 1,2-dipalmitoyl-3-phosphatidylserine (DPPS); palmitoyloleoyl phosphatidylethanolamine (POPE); and 1,2-dioleoyl-3-phosphatidylserine (DOPS).

18. The fusogenic liposome of claim 17, wherein said phospholipid is selected from the group consisting of DOPC, POPC, DMPC, DPPC, DOPE, POPE, DSPE, DMPE and DPPE.

19-20. (canceled)

21. The fusogenic liposome of claim 52, wherein the stabilizing moiety is PEG of molecular weight of about 106 Da to about 4 kDa.

22. The fusogenic liposome of claim 21, wherein PEG is of molecular weight of about 2 kDa.

23. The fusogenic liposome of claim 52, wherein said stabilizing moiety is connected to at least one of said lipid molecules via a cleavable peptide linker.

24. The fusogenic liposome of claim 52, wherein said first functional group of the specific binding pair is capable of forming a covalent bond with said complementary second functional group of said binding pair.

25. The fusogenic liposome of claim 24, wherein said first functional group of the specific binding pair is capable of forming a covalent bond with said complementary second functional group of said binding pair via a click chemistry reaction.

26. The fusogenic liposome of claim 24, wherein i) the first functional group of the specific binding pair is alkyne or phosphine, and the second functional group of said binding pair is azide, or vice versa; ii) the first functional group of the specific binding pair is cycloalkene, cycloalkyne, cyclopropane, isonitrile (isocyanide) or vinyl boronic acid, and the second functional group of said binding pair is tetrazine, or vice versa, iii) the first functional group of the specific binding pair is alkyne or maleimide, and the second functional group of said binding pair is thiol, or vice versa; iv) the first functional group of the specific binding pair is conjugated diene, and the second functional group of said binding pair is substituted alkene, or vice versa; v) the first functional group of the specific binding pair is alkene, alkyne or copper acetylide, and the second functional group of said binding pair is nitrone, or vice versa; vi) the first functional group of the specific binding pair is aldehyde or ketone, and the second functional group of said binding pair is alkoxyamine, hydroxylamine, hydrazine or hydrazide, or vice versa; or vii) the first functional group of the specific binding pair is aldehyde, ketone, isothiocyanate, carboxylic acid or a derivative thereof, and the second functional group of said binding pair is amine, or vice versa.

27. The fusogenic liposome of claim 26, wherein the specific binding pair is alkyne-azide.

28. The fusogenic liposome of claim 52, wherein said first functional group of the specific binding pair is capable of forming a non-covalent bond with said complementary second functional group of said binding pair.

29. The fusogenic liposome of claim 28, wherein the first functional group of the specific binding pair is biotin, and the second functional group of said binding pair is a biotin-binding peptide or biotin-binding protein, or vice versa.

30. The fusogenic liposome of claim 29, wherein said biotin-binding protein is selected from the group consisting of avidin, streptavidin and an anti-biotin antibody.

31. The fusogenic liposome of claim 29, wherein said biotin-binding peptide is selected from the group consisting of AEGEFCSWAPPKASCGDPAK (SEQ ID NO: 11), CSWRPPFRAVC (SEQ ID NO: 12), CSWAPPFKASC (SEQ ID NO: 13), and CNWTPPFKTRC (SEQ ID NO: 14).

32. The fusogenic liposome of claim 52, wherein the fusogenic liposome further comprises a first spacer between the lipid bilayer and the first functional group.

33. The fusogenic liposome of claim 54, wherein the immune system activating agent further comprises a second spacer between the immune system activating agent and the second functional group.

34. The fusogenic liposome of claim 32, wherein the first spacer is selected from the group consisting of PEG, (C₆-C₁₂)alkyl, phenolic, benzoic or naphthoic mono-, di- or tricarboxylic acid, tetrahydropyrene mono-, di- or tri-carboxylic acid, or salts thereof, cyclic ether, glutaric acid, succinate acid, muconic acid, adipic acid, pimelic acid, suberic acid, azelaic acid, and sebacic acid, and a peptide.

35. The fusogenic liposome of claim 34, wherein the first spacer is PEG of molecular weight of about 106 Da to about 4 kDa.

36. The fusogenic liposome of claim 35, wherein PEG is of a molecular weight of about 194 Da (PEG4).

37. The fusogenic liposome of claim 34, wherein the first spacer is (C₆-C₁₂)alkyl.

38. The fusogenic liposome of claim 52, wherein the fusogenic liposome further comprises cholesterol (CHO) or its derivatives.

39. The fusogenic liposome of claim 52, wherein the liposome has a size selected from the group consisting of up to 300 nm, up to 200 nm, from about 15 nm to about 200 nm, from about 20 nm to about 100 nm, from about 50 nm to about 150 nm, from about 50 nm to about 90 nm, from about 80 nm to about 100 nm, from about 110 nm to about 200 nm, and about 100 nm.

40-43. (canceled)

44. The fusogenic liposome of claim 81, wherein the fusogenic liposome comprises:
a. DOPC:DOTAP:DSPE-PEG2K:DOPE-PEG₄-N₃ or
DOPC:DOTAP:DSPE-PEG2K:DOPE-PEG₄-BCN; or
b. DMPC:Cholesterol:DMPE-PEG4-N3 or
DMPC:Cholesterol:DMPE-PEG4-BCN,
wherein PEG2K represents PEG having a molecular weight of about 2 kDa and PEG₄ represents PEG having a molecular weight of about 194 Da, and the relative molar amount of DOPC is up to about 80%, the relative molar amount of DOTAP is up to about 80%, the relative molar amount of DSPE-PEG2K is up to about 20%, the relative molar amount of DOPE-PEG4 is up to about 20%, the relative molar amount of HSPC is up to about 65%, the relative molar amount of Cholesterol is up to about 40%, and the relative molar amount of DMPC is up to about 70%, and the fusogenic liposome has a size of about 50 nm to about 300 nm.

45. The fusogenic liposome of claim 44, wherein the fusogenic liposome comprises:
(i) DOPC:DOTAP:DSPE-PEG2K:DOPE-PEG4-N3 or DOPC:DOTAP:DSPE-PEG2K:DOPE-PEG4-BCN, in the molar ratio 52.5:35:0.6:10, 52.5:35:1.25:10, 52.5:35:2.5:10, 52.5:35:5:10, 52.5:35:0.6:5, 52.5:35:1.25:5, 52.5:35:2.5:5, 52.5:35:5:5, 65:20:5:10, 50:35:5:10, 52.5:35:1.25:7, 52.5:35:1.25:5, or 52.5:35:2.5:7; or
(ii) DMPC:Chol:DMPE-PEG4-N3 or DMPC:Chol:DMPE-PEG4-BCN, in the molar ratio 60:35:5.

46. The fusogenic liposome of claim 45, wherein the fusogenic liposome comprises DOPC:DOTAP:DSPE-PEG2K:DOPE-PEG4-N3 in the molar ratio 52.5:35:2.5:5.

47. The fusogenic liposome of claim 44, wherein said T-cell activating agent is bound via said second functional group to the first functional group of at least one of said lipid molecules at the outer leaflet of the fusogenic liposome.

48. The fusogenic liposome of claim 44, wherein said T-cell activating agent is bound via said second functional group to the first functional group of at least one of said lipid molecules at the inner leaflet of the fusogenic liposome.

49. The fusogenic liposome of claim 44, wherein said T-cell activating agent is bound via said second functional group to the first functional group of at least one of said lipid molecules at both the inner and outer leaflet of the fusogenic liposome.

50. The fusogenic liposome of 52, wherein the melting temperature (Tm) of the liposome is below 45° C., at which the fusogenic liposome is maintained at a non-crystalline transition phase thereby providing membrane fluidity required for fusion of liposome with cell membranes.

51. The method of claim 1, wherein the cancer is selected from the group consisting of breast cancer, melanoma and lung cancer.

52. A fusogenic liposome comprising a lipid bilayer comprising a plurality of lipid molecules having 14 to 24 carbon atoms, wherein at least one of said lipid molecules further comprises a cationic group, a cationic natural or synthetic polymer, a cationic amino sugar, a cationic polyamino acid or an amphiphilic cancer-cell binding peptide; at least one of said lipid molecules further comprises a stabilizing moiety selected from the group consisting of polyethylene glycol (PEG), polypropylene glycol, polyvinyl alcohol, polyvinylpyrrolidone (PVP), dextran, a polyamino acid, methyl-polyoxazoline, polyglycerol, poly(acryloyl morpholine), and polyacrylamide, and wherein at least one of said lipid molecules is functionalised with a first functional group of a specific binding pair capable of binding to a complementary second functional group of said binding pair.

53. (canceled)

54. The fusogenic liposome of claim 52, further comprising an immune system activating agent functionalised with a complementary second functional group of said binding pair bound to said first functional group.

55-69. (canceled)

70. A method for preparation of a fusogenic liposome with an immune system activating agent bound at the outer leaflet, said method comprising the reaction of the fusogenic liposome of claim 52 with an immune system activating agent functionalised with a complementary second functional group of the binding pair, wherein said second functional group binds to said first functional group, thereby yielding said fusogenic liposome with said immune system activating agent bound at the outer leaflet.

71. A method for preparation of a fusogenic liposome with an immune system activating agent bound at both the inner and outer leaflet, said method comprising the steps of:
(i) reacting a plurality of the lipid molecules of claim 52, with an immune system activating agent functionalised with a second functional group of the binding pair, wherein said second functional group binds to said first functional group of said lipid molecules, thereby yielding the lipid molecules linked to the immune system activating agent; and
(ii) preparing said fusogenic liposome from said lipid molecules obtained in step (i),
thereby yielding the fusogenic liposome functionalised with said immune system activating agent bound at both the inner and outer leaflet.

72. A method for preparation of a fusogenic liposome with an immune system activating agent bound at the inner leaflet, said method comprising the steps of:
(i) preparation of liposomes in a solution comprising the lipid molecules of claim 52 and an immune system activating agent functionalised with a second functional group of the binding pair capable of binding to said first functional group of said lipid molecules, thereby encapsulating a fraction of said immune system activating agents;
(ii) removal of non-encapsulated immune system activating agent from the solution;
(iii) reaction of the lipid molecules with the encapsulated immune system activating agent inside the aqueous interior of the liposomes prepared in step (i), wherein said second functional group of said immune system activating agent binds to said first functional group of said lipid molecules,
thereby yielding the fusogenic liposome functionalised with said immune system activating agent bound at the inner leaflet.

73. The of claim 72, wherein said solution further comprises at least one oxidation-reduction catalyst.

74. The of claim 73, wherein said at least one oxidation-reduction catalyst is a copper (I) salt, which is removed in step (ii) in addition to the non-encapsulated immune system activating agent, and the reaction in step (iii) is a copper-dependent click chemistry reaction.

75. The method of claim 70, wherein the fusogenic liposomes have a size selected from the group consisting of up to 200 nm, from about 15 nm to about 200 nm, from about 20 nm to about 100 nm, from about 50 nm to about 150 nm, from about 50 nm to about 90 nm, from about 80 nm to about 100 nm, from about 110 nm to about 200 nm, and about 100 nm.

76. A kit comprising:
a. a first container comprising a fusogenic liposome of claim 52;
b. a second container comprising an immune system activating agent functionalised with a second functional group of the binding pair capable of binding to said first functional group of said lipid molecules; and
c. a pamphlet with instructions for a method for treating cancer comprising administering to a cancer patient the fusogenic liposome of (a) and subsequently the immune system activating agent of (b).

77. The fusogenic liposome of claim 54, wherein said immune system activating agent is an agent that recruits and activates immune effector cells.

78. The fusogenic liposome of claim 54, further including two or more different immune system activating agents, each one functionalised with a complementary second functional group of said binding pair bound to said first functional group.

79. The fusogenic liposome of claim 52, further comprising an immune system activating agent functionalised with a complementary second functional group of said binding pair bound to said first functional group,
wherein said at least one of said lipid molecules comprises a cationic group selected from the group consisting of 1,2-dioleoyl-3-trimethylammoniumpropane chloride (DOTAP), dioctadecylamidoglycylspermine (DOGS), 1,2-di-O-octadecenyl-3-trimethylammonium propane (DOTMA), dimethyldioctadecylammonium (18:0 DDAB), and N1-[2-((1S)-1-[(3-aminopropyl)amino]-4-[di(3-amino-propyl)amino]butyl-carboxamido)ethyl]-3,4-di[oleyloxy]-benzamide (MVL5),
said synthetic polymer is selected from the group consisting of polyethyleneimines (PEI) and poly(2-(dimethylamino)ethyl methacrylate,
said natural polymer is chitosan,
said amino sugar is glucosamine,
said cationic polyamino acid is selected from the group consisting of poly(L-lysine), poly(L-arginine), poly(D-lysine), poly(D-arginine), poly(L-ornithine) and poly(D-ornithine), or said amphiphilic cancer-cell binding peptide is selected from the group consisting of Cecropin A; Cecropin A 1-8; and cyclic CNGRC,
at least some of the lipids are phospholipids selected from the group consisting of a phosphatidylcholine, a phosphatidylethanolamine, a phosphatidylserine, a phosphatidic acid and any combination thereof, each one of which comprises one or two identical or different fatty acid residues, wherein the fatty acid residues in the phosphatidyl moiety are saturated, mono-unsaturated or poly-unsaturated and have a carbon chain length of 14, 15, 16, 17, 18, 19, 20, 21, 22, 23 or 24 carbons,
the stabilizing moiety is PEG of molecular weight of about 106 Da to about 4 kDa,
said first functional group of the specific binding pair is capable of forming a covalent bond with said complementary second functional group of said binding pair or said first functional group of the specific binding pair is capable of forming a non-covalent bond with said complementary second functional group of said binding pair,
the fusogenic liposome further comprises a first spacer between the lipid bilayer and the first functional group,
the liposome has a size selected from the group consisting of up to 200 nm, from about 15 nm to about 200 nm, from about 20 nm to about 100 nm, from about 50 nm to about 150 nm, from about 50 nm to about 90 nm, from about 80 nm to about 100 nm, from about 110 nm to about 200 nm, and about 100 nm, and
the melting temperature (Tm) of the liposome is below 45° C., at which the fusogenic liposome is maintained at a non-crystalline transition phase thereby providing membrane fluidity required for fusion of liposome with cell membranes.

80. The fusogenic liposome of claim 79, wherein
said immune system activating agent is bound via said second functional group to the first functional group of at least one of said lipid molecules at the outer leaflet, inner or both outer and inner of the fusogenic liposome;
the immune system activating agent is selected from the group consisting of a T-cell activating agent, a pro-inflammatory cytokine, a memory killer T-cell activating peptide, a soluble human leukocyte antigen (sHLA) presenting a viral peptide, and a super-antigen;
said at least one of said lipid molecules comprising a cationic group is DOTAP;
said phospholipid is selected from the group consisting of 1-palmitoyl-2-oleoyl-sn-glycero-3-phosphocholine (POPC) and 1,2-dioleoyl-3-phosphatidylethanolamine (DOPE); 1,2-dimyristoyl-3-phosphatidylcholine (DMPC); 1,2-distearoyl-3-phosphatidylcholine (DSPC); 1,2-dimyristoleoyl-sn-glycero-3-phosphocholine (14:1 (Δ9-Cis) PC); 1,2-dimyristelaidoyl-sn-glycero-3-phosphocholine (14:1 (Δ9-Trans) PC); 1,2-dipalmitoleoyl-sn-glycero-3-phosphocholine (16:1 (Δ9-Cis) PC); 1,2-dipalmitelaidoyl-sn-glycero-3-phosphocholine (16:1 (Δ9-Trans) PC); 1,2-dipetroselenoyl-sn-glycero-3-phosphocholine (18:1 (Δ6-Cis) PC); 1,2-dioleoyl-3-phosphatidylcholine (18:1 (49-Cis) PC (DOPC)); 1,2-dielaidoyl-sn-glycero-3-phosphocholine (18:1 (Δ9-Trans) PC); 1,2-dilinoleoyl-sn-glycero-3-phosphocholine (18:2 (Cis) PC (DLPC)); 1,2-dilinolenoyl-sn-glycero-3-phosphocholine (18:3 (Cis) PC); 1,2-dieicosenoyl-sn-glycero-3-phosphocholine (20:1 (Cis) PC); 1,2-diarachidonoyl-sn-glycero-3-phosphocholine (20:4 (Cis) PC); 1,2-didocosahexaenoyl-sn-glycero-3-phosphocholine (22:6 (Cis) PC); 1,2-dierucoyl-sn-glycero-3-phosphocholine (22:1 (Cis) PC); 1,2-dinervonoyl-sn-glycero-3-phosphocholine (24:1 (Cis) PC); 1,2-dimyristoyl-3-3-phosphatidylethanolamine (DMPE); 1,2-dipalmitoyl-3-phosphatidylethanolamine (DPPE); dipalmitoylphosphatidylcholine (DPPC); 1,2-dioleoyl-3-phosphatidylethanolamine (DOPE); 1,2-distearoyl-3-phosphatidylethanolamine (DSPE); 1,2-dimyristoyl-3-phosphatidylserine (DMPS); 1,2-dipalmitoyl-3-phosphatidylserine (DPPS); palmitoyloleoyl phosphatidylethanolamine (POPE); and 1,2-dioleoyl-3-phosphatidylserine (DOPS);
the stabilizing moiety is PEG of molecular weight of about 2 kDa;
said first functional group of the specific binding pair is capable of forming a covalent bond with said complementary second functional group of said binding pair via a click chemistry reaction or i) the first functional group of the specific binding pair is alkyne or phosphine, and the second functional group of said binding pair is azide, or vice versa; ii) the first functional group of the specific binding pair is cycloalkene, cycloalkyne, cyclopropane, isonitrile (isocyanide) or vinyl boronic acid, and the second functional group of said binding pair is tetrazine, or vice versa; iii) the first functional group of the specific binding pair is alkyne or maleimide, and the second functional group of said binding pair is thiol, or vice versa; iv) the first functional group of the specific binding pair is conjugated diene, and the second functional group of said binding pair is substituted alkene, or vice versa; v) the first functional group of the specific binding pair is alkene, alkyne or copper acetylide, and the second functional group of said binding pair is nitrone, or vice versa; vi) the first functional group of the specific binding pair is aldehyde or ketone, and the second functional group of said binding pair is alkoxyamine, hydroxylamine, hydrazine or hydrazide, or vice versa; or vii) the first functional group of the specific binding pair is aldehyde, ketone, isothiocyanate, carboxylic acid or a derivative thereof, and the second functional group of said binding pair is amine, or vice versa;
the first functional group of the specific binding pair capable of forming a non-covalent bound is biotin, and the second functional group of said binding pair is its binding-partner selected from the group consisting of a biotin-binding peptide and a biotin-binding protein, or vice versa;
the immune system activating agent further comprises a second spacer between the immune system activating agent and the second functional group; and
the first or second spacer is selected from the group consisting of PEG, (C₆-C₁₂)alkyl, phenolic, benzoic or naphthoic mono-, di- or tricarboxylic acid, tetrahydropyrene mono-, di- or tri-carboxylic acid, or salts thereof, cyclic ether, glutaric acid, succinate acid, muconic acid, adipic acid, pimelic acid, suberic acid, azelaic acid, and sebacic acid, and a peptide.

81. The fusogenic liposome of claim 80, wherein
the immune system activating agent is a T-cell activating agent;
said phospholipid is selected from the group consisting of DOPC, POPC, DMPC, DPPC, DOPE, POPE, DSPE, DMPE and DPPE;
the specific binding pair is alkyne-azide;
said biotin-binding protein is selected from the group consisting of avidin, streptavidin and an anti-biotin antibody, or said biotin-binding peptide is selected from the group consisting of AEGEFCSWAPPKASCGDPAK (SEQ ID NO: 11), CSWRPPFRAVC (SEQ ID NO: 12), CSWAPPFKASC (SEQ ID NO: 13), and CNWTPPFKTRC (SEQ ID NO: 14); and
the first or second spacer is PEG of molecular weight of about 106 Da to about 4 kDa or the first or second spacer is (C₆-C₁₂)alkyl.

82. The fusogenic liposome of claim 81, wherein the T-cell activating agent is selected from the group consisting of anti-CD3 antibody, an anti-CD8 antibody, an anti-NKG2D antibody, or a combination thereof, an antibody capable of binding both CD3 and CD8 and an antibody capable of binding both CD3 and NKG2D; and the spacer is PEG of a molecular weight of about 194 Da (PEG4).

83. The fusogenic liposome of claim 33, wherein the second spacer is selected from the group consisting of PEG, (C₆-C₁₂)alkyl, phenolic, benzoic or naphthoic mono-, di- or tricarboxylic acid, tetrahydropyrene mono-, di- or tri-carboxylic acid, or salts thereof, cyclic ether, glutaric acid, succinate acid, muconic acid, adipic acid, pimelic acid, suberic acid, azelaic acid, and sebacic acid, and a peptide.

84. The fusogenic liposome of claim 83, wherein the second spacer is PEG of molecular weight of about 106 Da to about 4 kDa.

85. The fusogenic liposome of claim 84, wherein PEG is of a molecular weight of about 194 Da (PEG4).

86. The fusogenic liposome of claim 83, wherein the second spacer is (C₆-C₁₂)alkyl.
